(19) United States
(12) Patent Application Publication
(10) Pub. No.: US 2004/0072738 A1
(43) Pub. Date: Apr. 15, 2004

(54) Analogues of thiocoraline and be-22179

(76) Inventor: Dale L Boger, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 10/433,178

(22) PCT Filed: Dec. 21, 2001
(86) PCT No.: PCT/US01/50324

(30) Foreign Application Priority Data
Dec. 21, 2000 (US) 60257762

(51) Int. Cl. A61K 38/12; C07K 7/64

(52) U.S. Cl. 514/10; 530/317

(57) ABSTRACT

A process for the total synthesis of thiocoraline and BE-22179 establishes the relative and absolute stereochemistry of these compounds and enables the construction and characterization of a series of related analogues. The mechanism for the bioactivity of thiocoraline, BE-22179 and their related analogues is charaterized. Thiocoraline, BE-22179, and their related analogues are disclosed to bind to DNA by high-affinity bisintercalation and are disclosed to exhibit exceptional cytotoxic activity.

### FIELD OF INVENTION

The invention relates to having antitumor antibiotics. More particularly, the invention relates to analogs of thiocoraline and BE-22179 having DNA bisintercalation and antitumor antibiotic activities.

### BACKGROUND

Thiocoraline (1, FIG. 1) is a potent antitumor antibiotic (Romeo, F., et al., J. Antibiot 1997, 50, 734; Perez Baz, et al., J. Antibiot. 1997, 50, 738; Perez Baz, J., et al., PCT Int. Appl., WO952773, 1995; Chem. Abst. 1995, 124, 115561) isolated from Micromonospora sp. L-13-ACM2-092. It constitutes the newest member of the two-fold symmetric bicyclic octadepsipeptides which include the antitumor antibiotics BE-22179 (Okada, H., et al., J. Antibiot. 1994, 47, 129) (2), triostin A (Shoji, J., et al., J. Antibiot. 1961, 14, 335; Shoji, J., et al., J. Org. Chem. 1965, 30, 2772; Otsuka, H., et al., Tetrahedron 1967, 23, 1535; Otsuka, H., et al., J. Antibiot 1976, 29, 107) (3), and echinomycin (Corbaz, R., et al., Helv. Chim. Acta 1957, 40, 199; Keller-Schierlein, W., et al., Helv. Chim. Acta 1957, 40, 205; Keller-Schierlein, W., et al., Helv. Chim. Acta 1959, 42, 305; Martin, D. G., et al., J. Antibiot. 1975, 28, 332; Dell, A., et al., J. Am. Chem. Soc. 1975, 97, 2497) (4), which bind to DNA with bisintercalation (Waring, M. J., et al., Nature 1974, 252, 653; Wang, A. H.-J., et al., Science 1984, 225, 1115; Quigley, G. J., et al., Science 1984, 232, 1255; Yoshinari, T., et al., Jpn. J. Cancer Res. 1994, 85, 550). Unlike BE-22179, thiocoraline does not inhibit DNA topoisomerase I or II, but it does inhibit DNA polymerase α at concentrations that inhibit cell cycle progression and clonogenicity (Erba, E., et al., British J. Cancer 1999, 80, 971; Yoshinari, T., et al., Jpn. J. Cancer Res. 1994, 85, 550). It was found to unwind double-stranded DNA (Erba, E., et al., British J. Cancer 1999, 80, 971; Yoshinari, T., et al., Jpn. J. Cancer Res. 1994, 85, 550), and was suggested to bind to DNA with bisintercalation analogous to triostin, echinomycin, and members of the larger cyclic decadepsipeptides including sandramycin (FIG. 2) (Isolation: Matson, J. A., et al., J. Antibiot. 1989, 42, 1763; Total synthesis: Boger, D. L., et al., J. Am. Chem. Soc. 1993, 115, 11624; Boger, D. L., et al., J. Am. Chem. Soc. 1996, 118, 1629; Boger, D. L., et al., Bioorg. Med. Chem. 1999, 7, 315; Boger, D. L., et al., Bioorg. Med. Chem. 1998, 6, 85), the luzopeptins (Boger, D. L., et al., Bioorg. Med. Chem. 1999, 7, 315; Boger, D. L., et al., Bioorg. Med. Chem. 1998, 6, 85; Isolation: Konishi, M., et al., J. Antibiot. 1981, 34, 148; Structure and stereochemistry: Arnold, E., et al., J. Am. Chem. Soc. 1981, 103, 1243; Total synthesis (luzopeptins A-C): Boger, D. L., et al., J. Am. Chem. Soc. 1999, 121, 1098; Boger, D. L., et al., J. Am. Chem. Soc. 1999, 121, 11375; Luzopeptin E2: Ciufolini, M. A., et al., J. Heterocyclic Chem. 1999, 36, 1409; Ciufolini, M. A., et al., Angew. Chem., Int Ed. 2000, 39, 2493), and the quinoxapeptins (Isolation: Lingham, R. B, et al., J. Antibiot. 1996, 49, 253; Total synthesis: Boger, D. L., et al., Jin, Q. Angew. Chem., Int Ed. 1999, 38, 2424). The initial studies on thiocoraline as well as BE-22179 established their two-dimensional structures but not their relative and absolute stereochemistry (Romeo, F., et al., J. Antibiot. 1997, 50, 734; Perez Baz, J, et al., J. Antibiot. 1997, 50, 738; Perez Baz, J., et al., PCT Int. Appl., WO952773, 1995; Chem. Abst 1995, 124, 115561; Okada, H., et al., J. Antibiot. 1994, 47, 129). Triostin A and echinomycin possess a D-stereochemistry at the α-position of the amide linkage to the quinoxaline chromophore (D-Ser) and L-stereochemistry at the remaining stereogenic centers. It has been shown that the analogous centers of sandramycin (Isolation: Matson, J. A., et al., J. Antibiot. 1989, 42, 1763; Total synthesis: Boger, D. L., et al., J. Am. Chem. Soc. 1993, 115, 11624; Boger, D. L., et al., J. Am. Chem. Soc. 1996, 118, 1629) and the quinoxapeptins (Isolation: Lingham, R. B., et al., J. Antibiot. 1996, 49, 253; Total synthesis: Boger, D. L., et al., Angew. Chem., Int. Ed. 1999, 38, 2424), like the luzopeptins (Isolation: Konishi, M., et al., J. Antibiot. 1981, 34, 148; Structure and stereochemistry: Arnold, E., et al., J. Am. Chem. Soc. 1981, 103, 1243; Total synthesis (luzopeptins A-C): Boger, D. L., et al., J. Am. Chem. Soc. 1999, 121, 1098; Boger, D. L., et al., J. Am. Chem. Soc. 1999, 121, 11375; Luzopeptin E2: Ciufolini, M. A., et al., J. Heterocyclic Chem. 1999, 36, 1409; Ciufolini, M. A., et al., Angew. Chem., Int. Ed. 2000, 39, 2493), also incorporate D-Ser. Moreover, it was reported that a synthetic analog of 3 possessing an all L-stereochemistry showed no appreciable DNA binding (Ciardelli, T. L., et al., J. Am. Chem. Soc. 1978, 100, 7684).

What is needed is a total synthesis of thiocoraline and of BE-22179. What is needed is the establishment of the relative and absolute stereochemistry of these compounds (Boger, D. L., et al., J. Am. Chem. Soc. 2000, 122, 2956) and a characterization of their activities. What is needed is the design and preparation of analogues.

### SUMMARY

Full details of the total synthesis of thiocoraline and BE-22179, C2 symmetric bicyclic octadepsipeptides possessing two pendant 3-hydroxyquinoline chromophores, are described and served to establish their relative and absolute stereochemistry. Key elements of the approach include the late stage introduction of the chromophore, symmetrical tetrapeptide coupling, macrocyclization of the 26-membered octadepsipeptide conducted at the single secondary amide site following disulfide formation, and a convergent assemblage of the tetradepsipeptide with introduction of the labile thiol ester linkage in the final coupling reaction under near racemization free conditions. By virtue of the late stage introduction of the chromophore and despite the challenges this imposes on the synthesis, this approach provides ready access of a range of key chromophore analogues. Thiocoraline and BE-22179 were shown to bind to DNA by high-affinity bisintercalation analogous to echinomycin, but with little or no perceptible sequence selectivity. Both 1 and 2 were found to exhibit exceptional cytotoxic activity (IC₅₀=200 and 400 pM, respectively, L1210 cell line) comparable to echinomycin and one analogue, which bears the luzopeptin chromophore, was also found to be a potent cytotoxic agent.

One aspect of the invention is directed to a compound represented by the following structure:

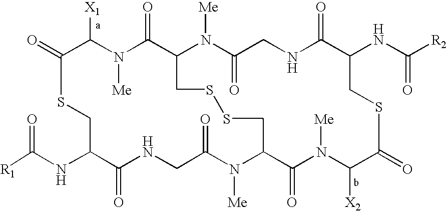

In the above structure, X₁ and X₂ can be either ═CH₂ or —CH₂SMe. R₁ and R₂ are selected from the group consisting of hydrogen, Cbz, FMOC, and radicals represented by the following structure:

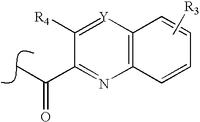

In the above structure, Y can be either C and N; R₃ can be either absent or —O(C1-C6 alkyl); and R₄ can be either hydrogen or hydroxyl. However, the following provisos pertain: if X, is ═CH₂, then “a” represents a double bond and neither R₁ nor R₂ is hydrogen; if X₁ is —CH₂SMe, then “a” represents a single bond; if X₂ is ═CH₂, then “b” represents a double bond and neither R₁ nor R₂ is hydrogen; if X₁ is —CH₂SMe, then “b” represents a single bond; and if R₃ is absent, then Y is N or R₄ is hydrogen. A preferred embodiment of this aspect of the invention is represented by the following diastereomeric structure:

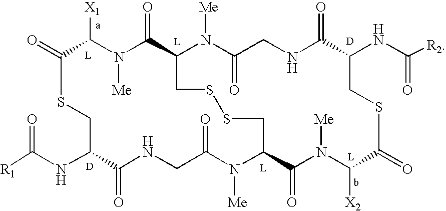

A subgenus of this aspect of the invention is represented by the following diastereomeric structure:

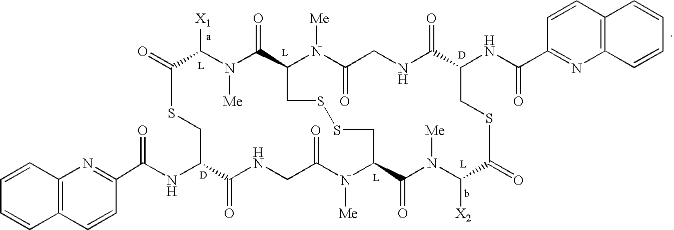

Preferred species of this subgenus are represented by the following diastereomeric structures:

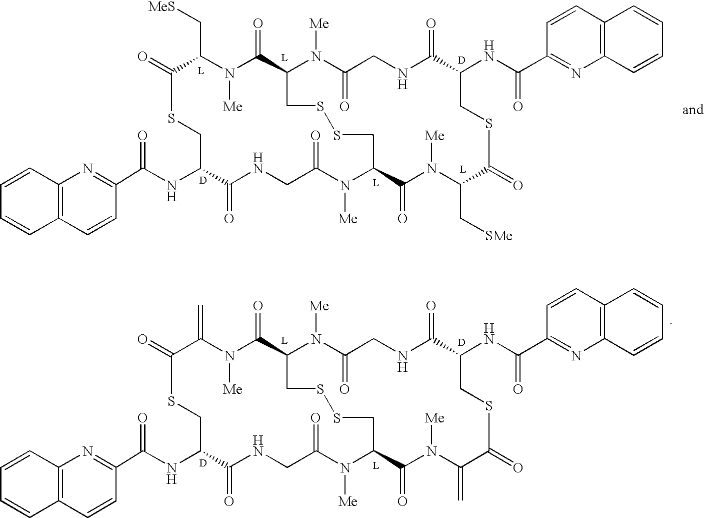

A second subgenus of this aspect of the invention is represented by the following diastereomeric structure:

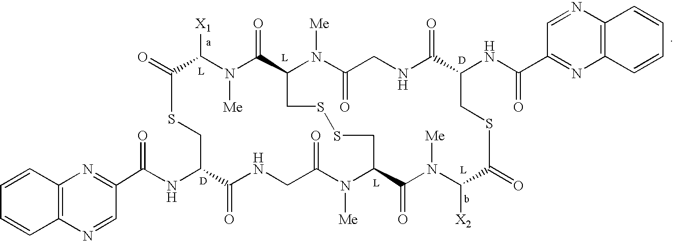

Preferred species of this second subgenus are represented by the following diastereomeric structures

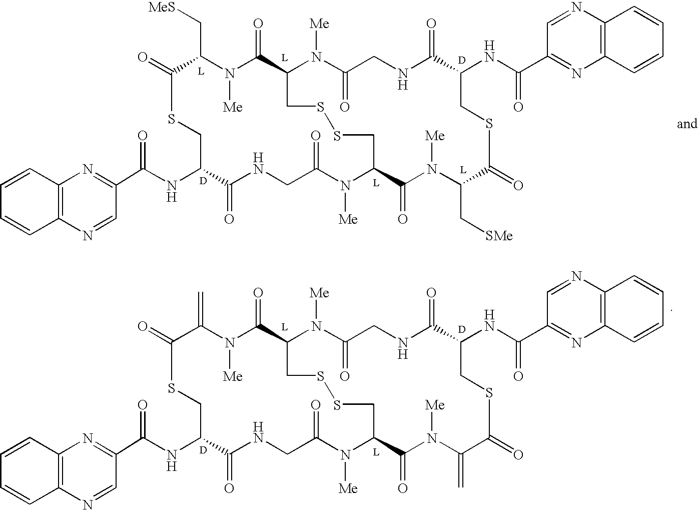

A third subgenus of this aspect of the invention is represented by the following diastereomeric structures:

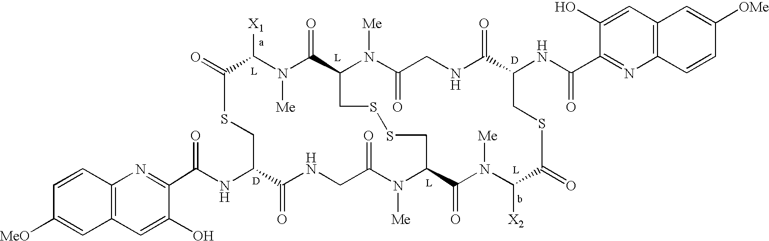

Preferred species of this third subgenus are represented by the following diastereomeric structures:

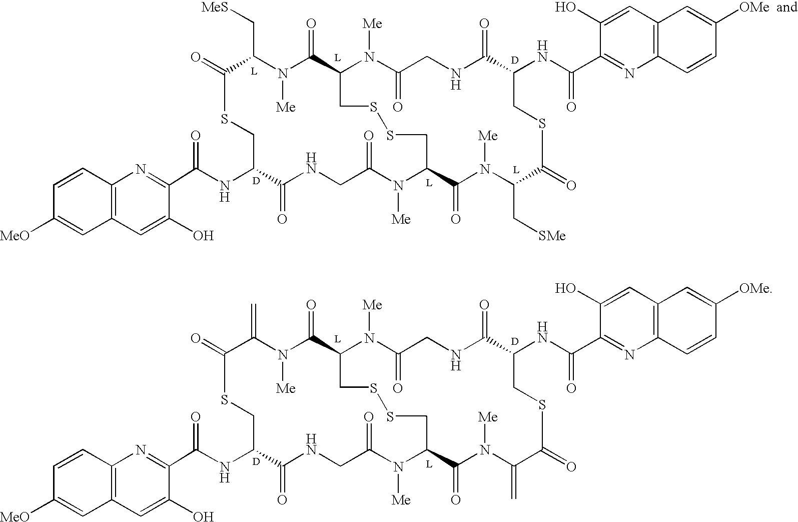

A fourth subgenus of this aspect of the invention is represented by the following diastereomeric structure:

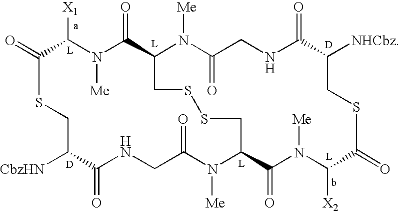

Preferred species of this fourth subgenus are represented by the following diastereomeric structures:

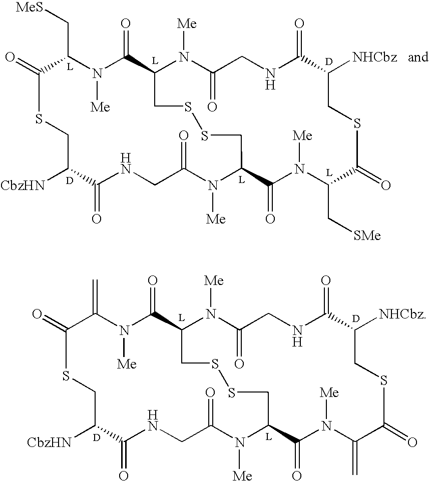

A fifth subgenus of this aspect of the invention is represented by the following diastereomeric structure:

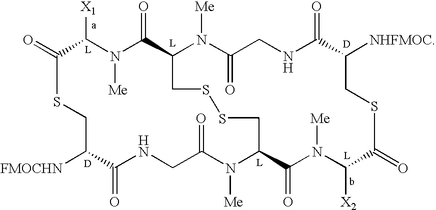

Preferred species of this fifth subgenus are represented by the following diastereomeric structures:

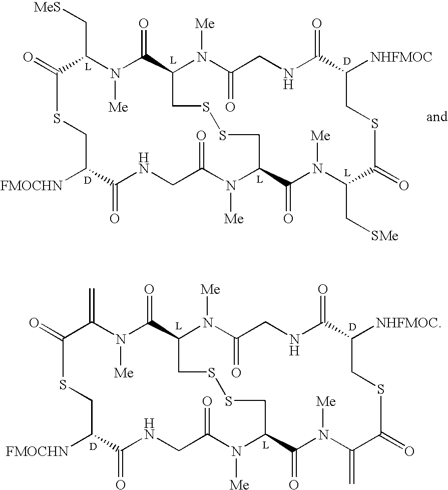

A further preferred species of this aspect of the invention is represented by the following diastereomeric structure:

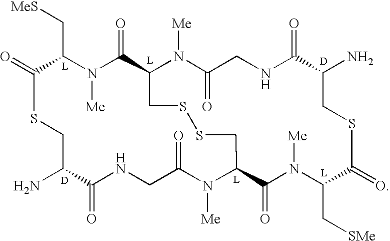

Another aspect of the invention is directed to a process for killing a cancer cell. The process comprises the step of contacting said cancer cell with a composition containing a concentration of thiocoraline, BE-22179, or any of the analogues of thiocoraline, BE-22179 described above, the concentration being sufficient to be cytotoxic with respect to said cancer cell.

Another aspect of the invention is directed to a process for binding thiocoraline, BE-22179, or or any of the analogues of of thiocoraline, BE-22179 described above to a deoxyoligonucleotide or to a deoxypolynucleotide. The process comprises the step of binding the thiocoraline, BE-22179, or any of the analogues of of thiocoraline, BE-22179 described above to such deoxyoligonucleotide or to such deoxypolynucleotide by bisintercalation.

Another aspect of the invention is directed to a process for synthesizing an advanced intermediate. The process comprises the step of cyclizing a first intermediate represented by the following structure:

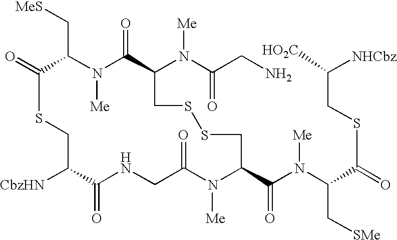

for producing the advanced intermediate represented by the following structure:

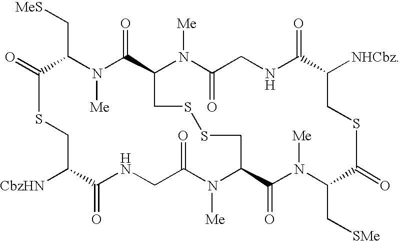

### BRIEF DESCRIPTION OF FIGURES

FIG. 1 illustrates the structures of thiocoraline (1), BE-22179 (2), triostin A (3) and echinomycin (4).

FIG. 2 illustrates the structures of members of the larger cyclic decadepsipeptides including sandramycin, the luzopeptins, and the quinoxapeptins.

FIG. 3 illustrates a scheme showing a convergent assemblage of key tetradepsipeptide 16 from tripeptide 15 and N-Cbz-D-Cys-OTce (11) along with the preparation of the three suitably functionalized Cys residues found in 1.

FIG. 4 illustrates a scheme for the synthesis of 2, 26, 27 and 28.

FIG. 5 illustsrates a scheme showing the series of steps required for the macrocyclization of 31.

FIG. 6 illustrates an approach in which the pendant chromophore was introduced at the initial stages of the synthesis.

FIG. 7 illustrates two plots of fluorescence vs. the DNA to drug ratio and the resulting Scatchard plot for each.

FIG. 8 illustsrates a table of comparative DNA binding properties.

FIG. 9 illustrates an electrophoresis gel of DNase footprinting of echinomycin bound to w794 DNA.

FIG. 10 illustrates an electrophoresis gel of DNase footprinting of thiocoraline bound to w794 DNA.

FIG. 11 illustrates a series of three electrophoresis agarose gels in which thiocoraline, echinomycin, BE-22179, and 27 are tested for their ability to uncoil DNA.

FIG. 12 illustrates a table showing that thiocoraline binds to DNA with high affinity, but with little or no selectivity.

FIG. 13 illustrate a table summarizing the biological activity of the compounds synthesized and similar natural compounds.

### DETAILED DESCRIPTION

Key elements of the approach include the late stage introduction of the chromophore, symmetrical tetrapeptide coupling, macrocyclization of the 26-membered octadepsipeptide conducted at the single secondary amide site following disulfide formation, and a convergent assemblage of the tetradepsipeptide with introduction of the labile thiol ester linkage in the final coupling reaction under near racemization free conditions. By virtue of the late stage introduction of the chromophore and despite the challenges this imposes on the synthesis because of a potential intramolecular S—N acyl transfer with cleavage of the macrocyclic thiol ester, this approach provided ready access to a range of chromophore analogues.

Tetradepsipeptide Synthesis.

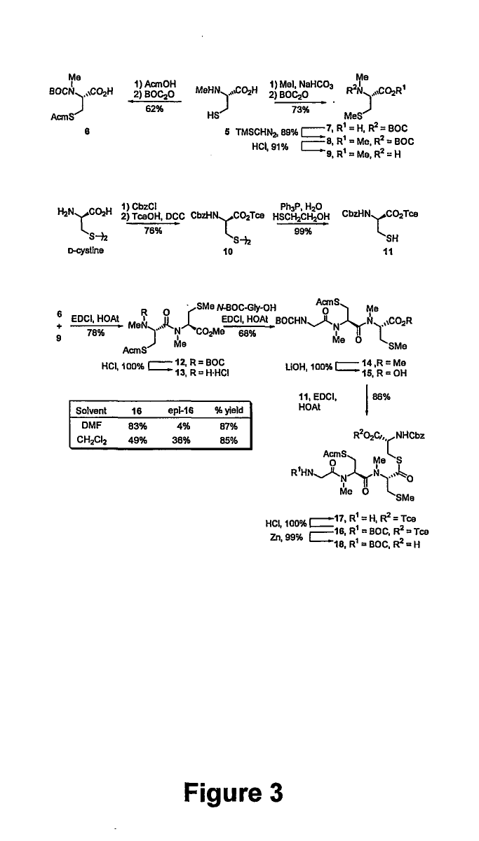

The convergent assemblage of key tetradepsipeptide 16 from tripeptide 15 and N-Cbz-D-Cys-OTce (11) along with the preparation of the three suitably functionalized Cys residues found in 1 are summarized in FIG. 3. Sequential S- and N-protection of N-Me-Cys-OH (5) (Blondeau, P., et al., Can. J. Chem. 1967, 45, 49) with an acetamidomethyl (Acm) group (1.5 equiv of N-hydroxymethylacetamide, H₂SO₄) and BOC group (BOC₂O, 62%) gave 6, the precursor to the bridging disulfide Cys residue. Selective S-methylation of N-Me-Cys-OH (5), (Blondeau, P., et al., Can. J. Chem. 1967, 45, 49) Mel, NaHCO₃) followed by BOC protection (BOC₂O, NaOH, 73%) provided 7. Esterification of 7 (TMSCHN₂, 89%) followed by BOC deprotection of 8 (3 M HCl-EtOAc, 91%) provided 9, the precursor to the second functionalized L-Cys residue. Alternative attempts to esterify 7 under basic conditions (Mel, NaHCO₃, DMF) or the exposure of 8 or 9 to tertiary amines (Et₃N, CH₂Cl₂) led to occasional extensive β-elimination of MeSH to provide the dehydro amino acid. Compound 11, constituting the chromophore bearing D-Cys residue, was prepared by the reduction of its disulfide precursor 10 (Ph₃P, 2-mercaptoethanol, 99%) which in turn was obtained by stepwise Cbz (CbzCl, NaHCO₃) and Tce (trichloroethanol, DCC, (DCC=dicyclohexylcarbodiimide; EDCl=1-(3-dimethylaminopropyl)-3-ethylcarbodiimide hydrochloride; HOBt=1-hydroxybenzotriazole; HOAt=1-hydroxy-7-azabenzotriazole) HOBt, 76%) protection of D-cystine. The esterification reaction with trichloroethanol proved sensitive to racemization and when conducted in the absence of HOBt (33% de vs 100% de) or in the presence of DMAP (33% de) led to extensive racemization. Coupling of 6 with 9 (EDCl, HOAt, 78%) provided 12 and slightly lower conversions was obtained with HOBt vs HOAt. BOC deprotection of 12 (3 M HCl-EtOAc, 100%), coupling with N-BOC-Gly-OH (EDCl, HOAt, 68%) and methyl ester hydrolysis of 14 (LiOH, 100%) provided 15.

The key thiol esterification reaction linking the D-cysteine derivative 11 and the tripeptide 15 was accomplished under near racemization free conditions with use of EDCl-HOAt (83%) in the absence of added base to afford the depsipeptide 16 (de 95:5). Much lower conversions were observed using DPPA (DPPA=diphenyl phosphorazidate; DEPC=diethyl phosphorocyanidate; Yamada, S., et al., J. Org. Chem. 1974, 39, 3302; Yokoyama, Y., et al., Chem. Pharm. Bull. 1977, 25, 2423) or DEPC and Et₃N due in part to competitive base-catalyzed formation of disulfide 10. Analogous to prior reports (DPPA=diphenyl phosphorazidate; DEPC=diethyl phosphorocyanidate; Yamada, S., et al., J. Org. Chem. 1974, 39, 3302; Yokoyama, Y., et al., Chem. Pharm. Bull. 1977, 25, 2423), near complete racemization was observed (16:epi-16=58:42) when the nonpolar solvent CH₂Cl₂ was used. In addition, the use of base in all reactions following formation of the thiol ester 16 was found to lead to competitive β-elimination or direct cleavage of the thiol ester and was necessarily avoided.

Cyclic Octadepsipeptide Formation and Completion of the Total Synthesis of Thiocoraline and BE-22179.

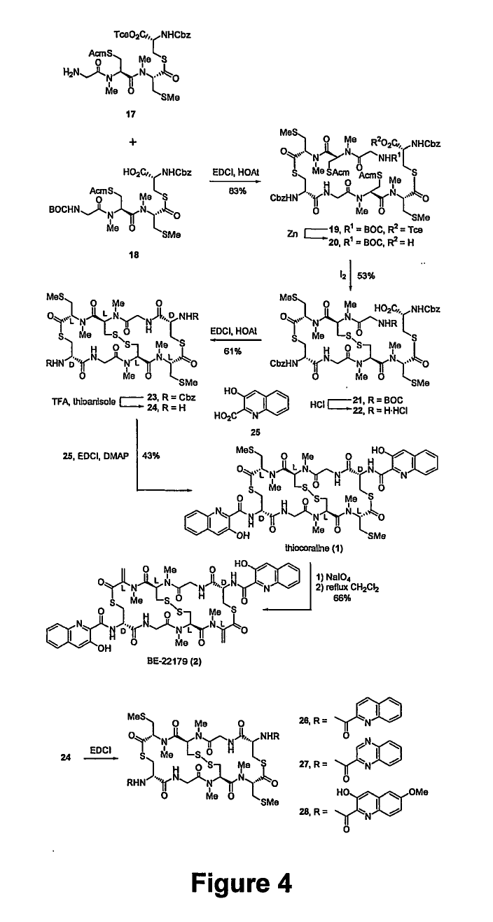

Linear octadepsipeptide formation was accomplished by deprotection of the amine (3 M HCl-EtOAc, 100%) and carboxylic acid (Zn, 90% aq. AcOH, 99%) of 16 to provide 17 and 18, respectively, which were coupled with formation of the secondary amide in the absence of added base (EDCl, HOAt, CH₂Cl₂, 83%) to obtain 19 (FIG. 4). Cyclization of 19 to provide the 26-membered cyclic octadepsipeptide 23 with ring closure conducted at the single secondary amide site was accomplished by sequential Tce ester deprotection (Zn, 90% aq. AcOH), disulfide bond formation (Kamber, B., et al., Helv. Chim. Acta 1980, 63, 899) (12, CH₂Cl₂-MeOH, 25° C., 0.001 M, 53% for 2 steps), and BOC deprotection (3 M HCl-dioxane) followed by treatment with EDCl-HOAt (0.001 M CH₂Cl₂, −20° C., 6 h, 61% for 2 steps). Reversing the N-BOC deprotection and disulfide bond formation steps in this 4-step sequence resulted in lower conversions (13% overall for 4 steps). To date, all attempts to effect ring closure followed by disulfide bond formation have not been successful. Even though the 26-membered ring macrocyclization reaction unconstrained by the disulfide bond proceeds exceptionally well (>50%), the subsequent disulfide bond formation (I₂, CH₂Cl₂—MeOH, 25° C.) within the confines of the 26-membered ring failed to occur. Thus, the order of steps enlisted for formation of 23 was not to improve macrocyclization via the constrained disulfide, but rather to permit disulfide bond formation. While it is possible this may be due to constraints within the macrocycle destabilizing the disulfide, the lack of similar observations with 3 and 4 suggest the origin of the difficulties may lie with competitive intramolecular cleavage of the adjacent thiol ester by the liberated bridging thiol within the 26-membered macrocycle.

Removal of the Cbz protecting group under mild conditions (Kiso, Y., et al., J. Chem. Soc., Chem. Commun. 1980, 101) (TFA-thioanisole, 25° C., 4 h) and coupling of the resulting amine 24 with 3-hydroxyquinoline-2-carboxylic acid (25, (Prepared from methyl 3-hydroxyquinoline-2-carboxylate (Boger, D. L., et al., J. Org. Chem. 1995, 60, 7369) by treatment with LiOH, THF-MeOH—H₂O 3/1/1, 25° C., 2 h (71%)) EDCl, DMAP, 43%) without protection of the chromophore phenol provided (−)-1, [α]²⁵_{D}-180 (c 0.11, CHCl₃) [lit¹ [α]²⁵_{D}-191 (c 1.1, CHCl₃)], identical in all respects with the properties reported for natural material (Romeo, F., et al., J. Antibiot. 1997, 50, 734; Perez Baz, J., et al., J. Antibiot. 1997, 50, 738; Perez Baz, J., et al., PCT Int. Appl., WO952773, 1995; Chem. Abst. 1995, 124, 115561). Under these conditions, a problematic intramolecular S—N acyl migration of the liberated amine with cleavage of the thiol ester was minimized. Treatment of 1 with NalO₄ served to provide the corresponding bis-sulfoxide as a mixture of diastereomers which was warmed in CH₂Cl₂ (reflux, 6 h, 66% overall) to promote elimination and provide (−)-BE-22179 (2), [α]_{D}²⁵-89 (c 0.01, CHCl₃) [lit (Okada, H., et al., J. Antibiot. 1994, 47, 129) [α]_{D}²⁵-94 (c0.44, CHCl₃)], identical all respects with the properties reported for the natural material (Okada, H., et al., J. Antibiot. 1994, 47, 129). The correlation of synthetic and natural 1 and 2 confirmed the two dimensional structure assignments and established their relative and absolute stereochemistries as those shown in FIG. 4.

Interestingly, both 23 and thiocoraline (1) as well as the related natural product analogues 26-28 adopt a single solution conformation that is observed by ¹H NMR in well defined spectra. That of synthetic 1 proved identical to the published ¹H NMR spectrum of natural 1 (Romeo, F., et al., J. Antibiot. 1997, 50, 734; Perez Baz, J., et al., J. Antibiot. 1997, 50, 738; Perez Baz, J., et al., PCT Int Appl., WO952773, 1995; Chem. Abst. 1995, 124, 115561). In contrast, BE-22179 exhibits a more complex, but still well defined, ¹H NMR spectrum consistent with its adoption of two unsymmetrical or four symmetrical conformers in near equal proportions. The NMe signals (2 NMe) and the two olefin signals (C═CHH) appear as eight, near 1:1, well resolved singlets in the ¹H NMR spectrum. Importantly, the ¹H NMR spectrum of synthetic 2 proved identical to that published for natural 2 (Okada, H., et al., J. Antibiot. 1994, 47, 129).

Alternative Approaches.

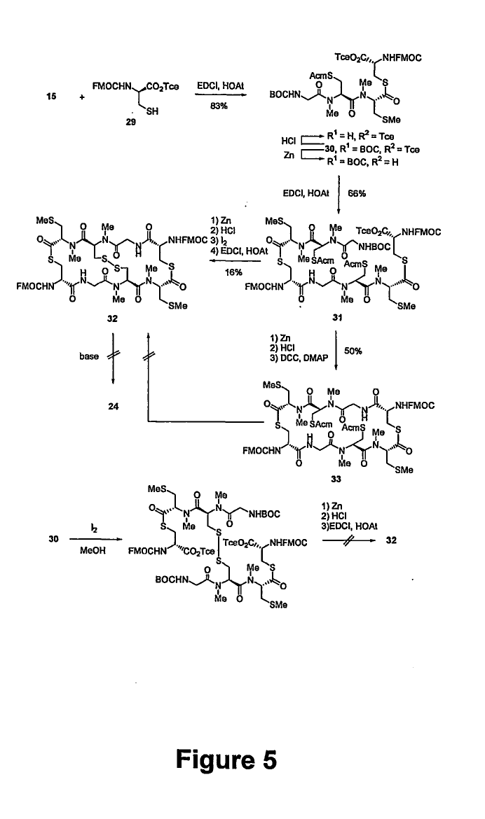

Prior to implementing the successful sequence, preliminary studies were first conducted enlisting an FMOC protecting group and basic deprotection conditions versus a Cbz protecting group on 23 (FIG. 5). Thus, tetradepsipeptide 30 and octadepsipeptide 31 were prepared by the procedures described for the synthesis of 16 and 19. Cyclization of 31 to provide the bridged 26-membered cyclic octadepsipeptide 32 was accomplished by sequential Tce ester deprotection (Zn, 90% aq. AcOH), BOC deprotection (3 M HCl-dioxane), and disulfide bond formation (I₂, CH₂Cl₂-MeOH, 25° C., 0.001 M) followed by treatment with EDCl-HOAt (0.001 M CH₂Cl₂, −20° C., 6 h, 16% for 4 steps). However, exposure of 32 to Et₂NH or piperidine led to decomposition of the macrocycle rather than clean FMOC deprotection. Alternative treatment of 32 with other amines including dicyclohexylamine, Et₃N, or DMAP also failed to provide the cyclic amine 24 which is attributed herein to the sensitivity of the thiol ester to nucleophiles, the competitive β-elimination induced by the deprotonation of the α-position of the Cys residues, and a potential intramolecular S—N acyl transfer to the liberated amine with cleavage of the thiol ester. However, efforts to trap the liberated amine in situ to obtain 1 directly (25, EDCl, DMAP) or a protected derivative of 24 (BOC₂₀ or CbzCl, Et₃N) were also unsuccessful.

Also examined was the approach in which the bridged 26-membered macrocycle is formed via simultaneous formation of both secondary amides. However, intermolecular disulfide bond formation (I₂, MeOH) and sequential deprotection of Tce and BOC group and the treatment of the resulting symmetrical disulfide with EDCl and HOAt gave complex mixtures of products including a range of oligomers and higher order macrocycles in which the formation of 32 was not observed (FIG. 5).

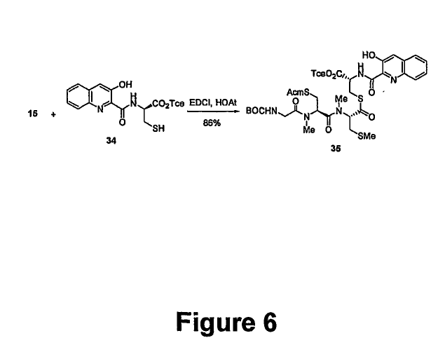

Finally, also examined was an approach in which the pendant chromophore was introduced at the initial stages of the synthesis. Thus, the coupling reaction of 15 and 34 (EDCl, HOAt, 86%) gave tetradepsipeptide 35 which possesses the substituted quinoline chromophore (FIG. 6). However, elimination of thiol ester was problematic under the conditions of BOC deprotection (HCl or 90% aq. TFA, 0° C.) or Tce ester hydrolysis (Zn, 90% aq. HOAc, 0° C.) and the following coupling reaction which gave only a trace of the desired linear octadepsipeptide. Presumably, this may be attributed to the increased acidity of the α-proton of the activated N-acyl-D-Cys derivative bearing an amide versus carbamate protecting group.

Analogue Synthesis.

The late stage generation of amine 24 followed by introduction of the pendant chromophore provided the opportunity to examine chromophore analogs of 1 and 2. Thus, the amine 24 was also coupled with quinoline-2-carboxylic acid, quinoxaline-2-carboxylic acid (which is the chromophore found in echinomycin and triostin A), and 3-hydroxy-6-methoxyquinoline-2-carboxylic acid (Isolation: Konishi, M., et al., J. Antibiot. 1981, 34, 148; Structure and stereochemistry: Arnold, E., et al., J. Am. Chem. Soc. 1981, 103, 1243; Total synthesis (luzopeptins A-C): Boger, D. L., et al., J. Am. Chem. Soc. 1999, 121, 1098; Boger, D. L., et al., J. Am. Chem. Soc. 1999, 121, 11375; Luzopeptin E2: Ciufolini, M. A., et al., J. Heterocyclic Chem. 1999, 36, 1409; Ciufolini, M. A., et al., Angew. Chem., Int Ed. 2000, 39, 2493; Boger, D. L., et al., J. Org. Chem. 1995, 60, 7369) (which is the chromophore found in the luzopeptins) to afford the key chromophore analogues 26-28 (FIG. 4). The corresponding analogues of 2 may be obtained by oxidation of 26-28 in a manner similar to the method shown in FIG. 4 for the oxidation of 1 to obtain 2.

DNA Binding Affinity.

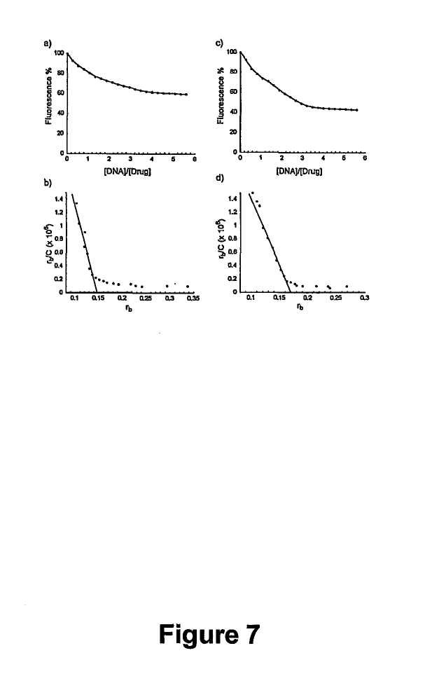

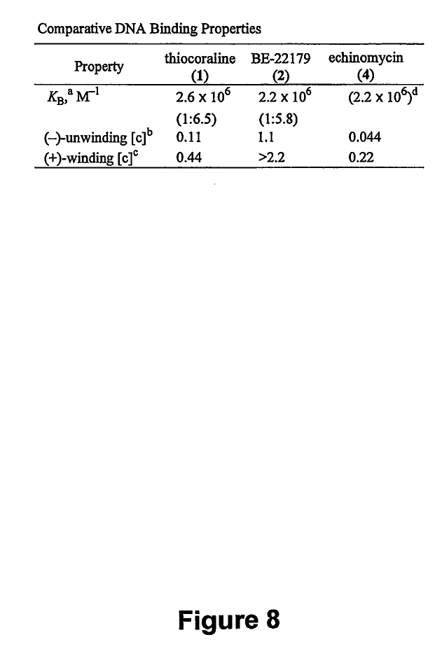

Apparent absolute binding constants and apparent binding site sizes were obtained by measurement of the fluorescence quenching upon titration of 1 and 2 with calf thymus (CT) DNA. The excitation and emission spectra for thiocoraline and BE-22179 were determined in aqueous buffer (Tris-HCl, pH 7.4, 75 mM NaCl). Both thiocoraline and BE-22179, which have the same chromophore, exhibited an intense fluorescence in solution with enhanced excitation (380 nm) and emission (510 nm) maxima which was quenched upon DNA binding. Moreover, the intensity of this fluorescence greatly facilitated the measurement of fluorescence quenching and allowed measurements to be carried out at low initial agent concentrations of 1-10 μM where the compounds are soluble. Analogous measurements with echinomycin could not be conducted because of its less intense fluorescence emission and low solubility. For the titrations, small aliquots of CT-DNA (320 μM in base pair) were added to 2 mL of a solution of the agent (2 μM) in Tris-HCl (pH 7.4), 75 mM NaCl buffer. Additions were carried out at 15-min intervals to allow binding equilibration. Scatchard analysis (Scatchard, G. Ann. N.Y. Acad. Sci. 1949, 51, 660) of the titration results was conducted using the equation r_{b}/c=Kn−Kr_{b}, where r_{b} is the number of molecules bound per DNA nucleotide phosphate, c is the free drug concentration, K is the apparent binding constant, and n is the number of the agent binding sites per nucleotide phosphate. A plot of r_{b}/c versus r_{b} gives the association constant (slope) and the apparent binding site size (x-intercept) for the agents (FIG. 7 and FIG. 8).

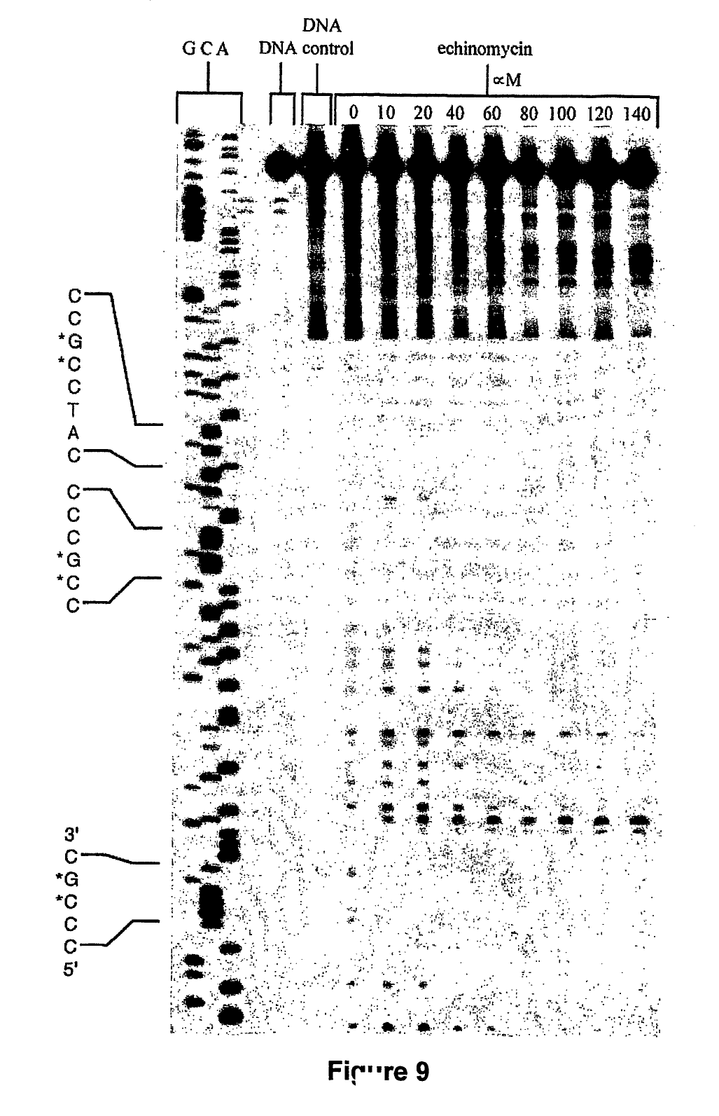

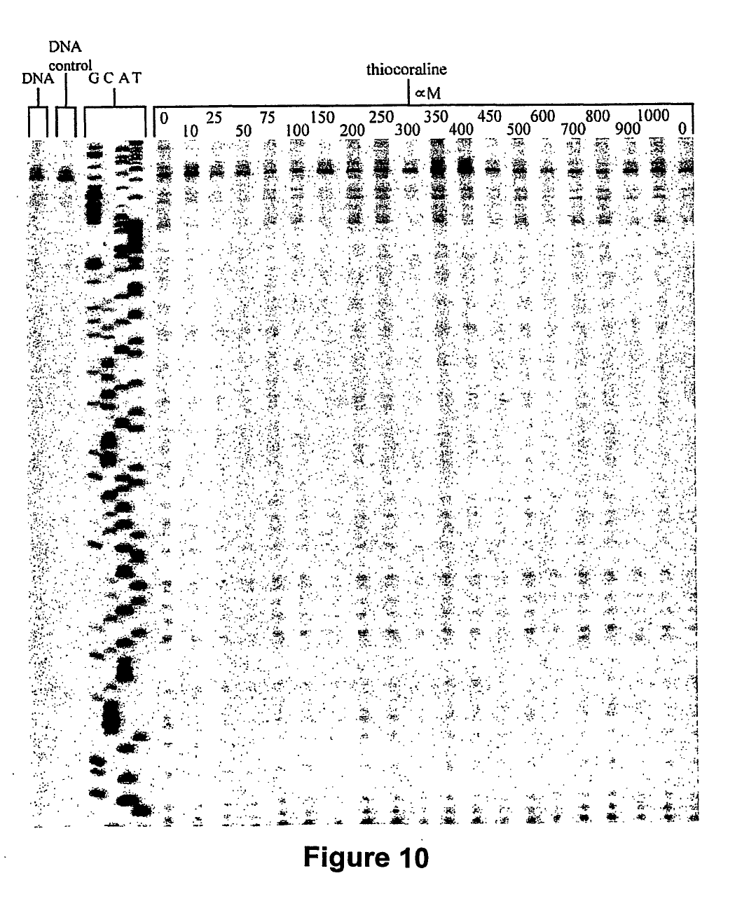

Thiocoraline was found to exhibit a relatively high affinity for duplex DNA (K_{B}=2.6×10⁶ M⁻¹) with a saturating stoichiometry of high affinity binding at a 1:6.5 agent to base pair ratio. BE-22179, which is structurally distinct possessing two exocyclic olefins, also displayed a similar affinity and binding site size with CT-DNA. The high affinity binding of one molecule per 5.86.5 base pairs approaches that of the saturated limit of 4 base pairs assuming bisintercalation spanning two base pairs suggesting thiocoraline and BE-22179 bind to DNA with limited selectivity among available sites. This proved consistent with attempts to establish a selectivity of DNA binding by DNase I (Galas, D. J., et al., Nucleic Acids Res. 1978, 5, 3157) and MTE footprinting (Tullius, T. D., et al., Methods Enzymol. 1987, 155, 537) on w794 DNA (Boger, D. L., et al., Tetrahedron 1991, 47, 2661), using protocols successfully applied to sandramycin (Isolation: Matson, J. A., et al., J. Antibiot. 1989, 42, 1763; Total synthesis: Boger, D. L., et al., J. Am. Chem. Soc. 1993, 115, 11624; Boger, D. L., et al., J. Am. Chem. Soc. 1996, 118, 1629) and echinomycin, which failed to reveal a distinguishable selectivity for 1 (FIGS. 9 and 10). Previous studies of sandramycin, the luzopeptins, and quinoxapeptins, which are larger symmetrical cyclic decadepsipeptides, revealed that they exhibit a higher affinity for CT-DNA (K^{B}=1.0-3.4×10⁷ M⁻¹). Since thiocoraline and BE-22179 possess the same chromophore as sandramycin (K^{B}=3.4×10⁷ M⁻¹), this indicates that the differing ability to bind duplex DNA arises from the cyclic depsipeptide, its ring size and differing peptide backbone and not the structure of the chromophore.

Similarly, echinomycin and triostin A bind to DNA by bisintercalation and are the most extensively studied natural products in these series. In contrast to sandramycin and the luzopeptins which bind 5′-PyPuPyPu sites and exhibit the highest affinity for 5′-CATG spanning a two base pair 5′-AT site (Boger, D. L., et al., Bioorg. Med. Chem. 1999, 7, 315; Boger, D. L., et al., Bioorg. Med. Chem. 1998, 6, 85; Isolation: Konishi, M., et al., J. Antibiot. 1981, 34, 148; Structure and stereochemistry: Arnold, E., et al., J. Am. Chem. Soc. 1981, 103, 1243; Total synthesis (luzopeptins A-C): Boger, D. L., et al., J. Am. Chem. Soc. 1999, 121, 1098; Boger, D. L., et al., J. Am. Chem. Soc. 1999, 121, 11375. Luzopeptin E2: Ciufolini, M. A., et al., J. Heterocyclic Chem. 1999, 36, 1409; Ciufolini, M. A., et al., Angew. Chem., Int Ed. 2000, 39, 2493), the quinoxalines bisintercalate preferentially at 5′-CG sites, e.g. 5′-GCGT or 5′-PuPyPuPy, also spanning two base pairs with each intercalation occurring at a PuPy vs PyPu step. The structural distinctions between 1 and 2 versus triostin A (3) are subtle. Beyond the different chromophores, they include the conservative side chain CH₂SCH₃ vs NMe-Val CH(Me)₂ alteration and the more significant Gly vs L-Ala (H vs Me) substitution, and the thioester vs ester (S vs 0) backbone alteration. Nonetheless, these changes abolished the DNA binding selectivity and, as shown below, may reduce the stability of the bisintercalation complexes.

Bifunctional Intercalation.

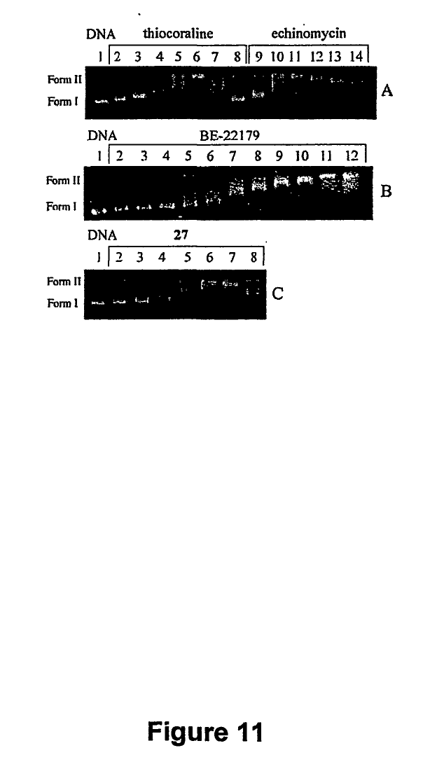

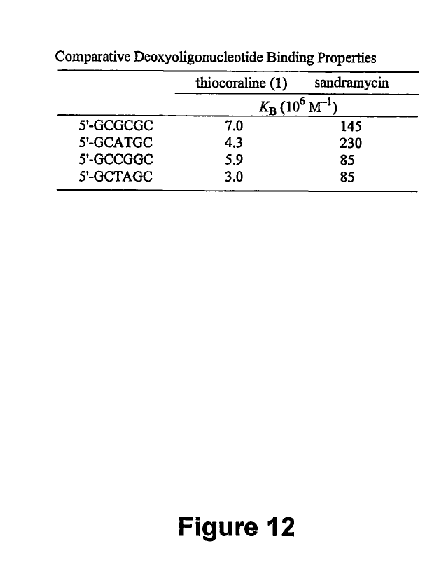

Confirmation that thiocoraline and BE-22179 bind to DNA with bisintercalation was derived from their ability to induce the unwinding of negatively supercoiled DNA. This was established by their ability to gradually decrease the agarose gel electrophoresis mobility of supercoiled ΦX174 DNA (unwinding) at increasing concentrations followed by a return to normal mobility (rewinding) at even higher concentrations. Under the conditions employed, echinomycin unwound ΦX174 DNA at a 0.044 agent/base pair ratio (FIG. 11 and FIG. 12). Thiocoraline completely unwound ΦX174 DNA at a higher 0.11 agent/base pair ratio, whereas BE-22179 required even higher concentrations producing the unwinding at an agent/base pair ratio of 1.1. Complete rewinding of the supercoiled DNA occurred at an agent/base pair ratio of 0.44 for thicoraline vs 0.22 for echinomycin whereas BE-22179 failed to produce the rewinding of ΦX174 DNA at the concentrations examined. The thiocoraline analogue 27, which bears the quinoxaline chromophore of echinomycin, was found to behave in a manner indistinguishable from thiocoraline itself. Thus, the distinctions in 1 and 2 and echinomycin detected here appear to be related to the nature of the cyclic depsipeptide and not the structure of the chromophore. Under these conditions, ethidium bromide, a monointercalater, does not unwind supercoiled DNA although it can unwind supercoiled DNA under conditions which prevent dissociation of the bound agent during electrophoresis. Thus, the unwinding of negatively supercoiled DNA and the subsequent positive supercoiling of the DNA by thiocoraline and 27, indicative of bisintercalation, were similar although slightly less effective than echinomycin, whereas that of BE-22179 was substantially less effective. This suggests that BE-22179 binds with a smaller unwinding angle, with lower stability, or with faster off-rates than echinomycin and thiocoraline.

Also examined was the ability of 1 or 2 to cleave, alkylate, or cross-link DNA. In particular, the electrophilic unsaturation found in BE-22179 might be expected to serve as an alkylation site for covalent attachment to DNA, especially following bisintercalation binding. No evidence was found to suggest that either 1 or 2 cleave DNA in simple assays monitoring the conversion of supercoiled ΦX174 DNA (Form I) to relaxed (Form II) or linear (Form III) DNA under a range of conditions. Similarly, sequencing cleavage studies conducted with w794 DNA enlisting the thermal depurination and cleavage detection of adenine N3 or N7 or guanine N3 or N7 alkylation sites did not reveal alkylation by 2. However, these studies do not exclude alkylation at non thermally labile sites including the guanine C2 amine. Additional assays conducted with w794 DNA following established protocols (Boger, D. L., et al., Tetrahedron 1991, 47, 2661) provided no evidence of DNA interstrand cross-linking. These studies would detect both thermally labile and non thermally labile alkylation sites, but only those engaged in interstrand cross-linking. Given the C2 symmetric nature of 2, bisintercalation analogous to echinomycin and triostin A places the two electrophilic sites in positions to react only with the complementary strands of duplex DNA (interstrand DNA cross-linking) and would preclude intrastrand DNA cross-linking. Thus, these studies safely excluded DNA cross-linking by 2 even with reaction of non thermally labile sites (e.g. G C2 amine), but do not rule out monoalkylation events at non thermally labile sites.

DNA Binding Selectivity.

The preceding studies suggested that thiocoraline binds to DNA with high affinity, but with little or no selectivity. Consequently, the binding of 1 was examined with a set of four duplex deoxyoligonucleotides, 5′-GCXXGC-3′ where XX=TA, AT, GC, CG, incorporating the high affinity intercalation sites of the related bisintercalatiors echinomycin (5′-PuCGPy) (Corbaz, R., et al., Helv. Chim. Acta 1957, 40, 199; Keller-Schierlein, W., et al., Helv. Chim. Acta 1957, 40, 205; Keller-Schierlein, W., et al., Helv. Chim. Acta 1959, 42, 305; Martin, D. G., et al., J. Antibiot. 1975, 28, 332; Dell, A., et al., J. Am. Chem. Soc. 1975, 97, 2497), sandramycin (5′-CATG) (Boger, D. L., et al., Bioorg. Med. Chem. 1999, 7, 315; Boger, D. L., et al., Bioorg. Med. Chem. 1998, 6, 85), and the luzopeptins (5′-CATG) (Isolation: Konishi, M., et al., J. Antibiot. 1981, 34, 148; Structure and stereochemistry: Arnold, E., et al., J. Am. Chem. Soc. 1981, 103, 1243. Total synthesis (luzopeptins A-C): Boger, D. L., et al., J. Am. Chem. Soc. 1999, 121, 1098; Boger, D. L., et al., J. Am. Chem. Soc. 1999, 121, 11375; Luzopeptin E2: Ciufolini, M. A., et al., J. Heterocyclic Chem. 1999, 36, 1409; Ciufolini, M. A., et al., Angew. Chem., int Ed. 2000, 39, 2493). The binding constants were established by titration using the fluorescent quenching that is observed upon DNA binding. The excitation and emission spectra for thiocoraline and BE-22179 were recorded in aqueous buffer (Tris-HCl, pH 7.4, 75 mM NaCl). To minimize fluorescence decrease due to dissolution or photobleaching, the solutions were stirred in 4-mL cuvettes in the dark with the minimum exposure to the excitation beam necessary to obtain a reading. The titrations were carried out with a 15-min equilibration time after each deoxyoligonucleotide addition. Scatchard plots of thiocoraline binding to the deoxyoligonucleotides exhibited a downward convex curvature which is interpreted herein to indicate a high-affinity bisintercalation and a lower affinity binding potentially involving monointercalation. Using the model described by Feldman (Feldman, H. A. Anal. Biochem. 1972, 48, 317) which assumes one ligand with two binding sites, the curves were deconvoluted according to the equation

r_{b}/c=½[(K₁(n₁−r_{b})+K₂(n₂−r_{b}))+{square root over ((K₁(n₁−r_{b})−K₂(n₂−r_{b}))²+4K₁K₂n₁n₂])} (1)

where K₁ and K₂ represent the association constants for high- and low-affinity binding, and n₁ and n₂ represent the number of bound agents per duplex for the separate binding events. Scatchard plots of the data revealed 1:1 binding in each case. That of the high affinity binding is consistent with binding of a single molecule with bisintercalation surrounding a central two base pair site. A small preference was observed for GC-rich binding with 5′-GCGCGC and 5′-GCCGGC exhibiting the tightest binding, but the differences are small ranging from 3-7×10₆ M⁻¹ for the four deoxyoligonucleotides (FIG. 12). Thus, consistent with the results of footprinting and other related studies herein, the binding of 1 with the deoxyoligonucleotides exhibited little selectivity.

Biological Properties.

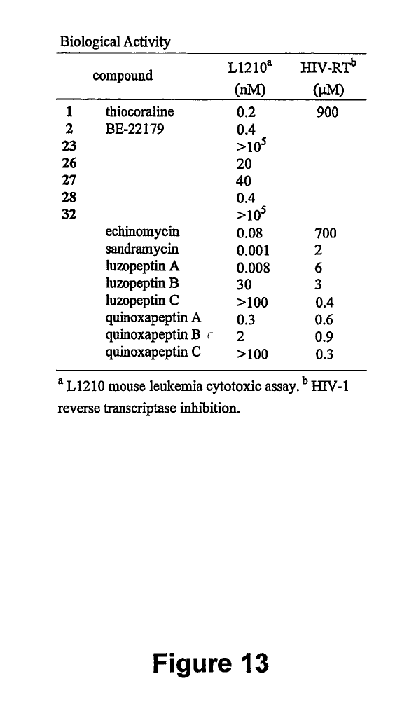

FIG. 13 summarizes the biological properties of echinomycin, thiocoraline, and BE-22179 along with those of precursor 23 and their analogues. Thiocoraline and BE-22179 exhibit exceptionally potent cytotoxic activity in the L1210 assays (IC₅₀=200 and 400 pM, respectively) being slightly less potent than echinomycin. Compounds 23 and 32 lacking both chromophores and containing the Cbz and FMOC protecting groups were inactive and >10⁵ times less potent than thiocoraline. Analogue 28, which bears the same chromophore as the luzopeptins, also exhibited potent activity while 26, lacking the quinoline C3 phenol, and 27, bearing the quinoxaline chromophore of echinomycin and triostin A, exhibited less potent cytotoxic activity. In addition, thiocoraline, like echinomycin, was found to be only a weak inhibitor of HIV-1 reverse transcriptase.

Most notable of these observations is that both thiocoraline and BE-22179 are exceptionally potent cytotoxic agents joining the small group of compounds that exhibit IC₅₀'s at subnanomolar or low picomolar concentrations (200-400 pM).

### EXPERIMENTAL SECTION

N-BOC-NMe-L--Cys(Acm)-OH (6).

A solution of NMe-L--Cys-OH hydrochloride salt (5, 1.35 g, 10.0 mmol) and acetamidomethanol (13.4 g, 15 mmol) in water (5 mL) was treated with conc. HCl (0.64 mL) and the reaction mixture was stirred at 25° C. for 12 h. The reaction mixture was concentrated in vacuo. The residue was dissolved in 100 mL of THF-H₂O (1:1) and the resulting solution was brought to pH 8 by adding 1 N aqueous NaOH. Di-tert-butyl dicarbonate (2.62 g, 12.0 mmol) was added and the reaction mixture was stirred at 25° C. for 12 h maintaining a pH 8. The reaction mixture was poured onto 1 N aqueous HCl (150 mL) and extracted with CHCl₃ (3×100 mL). The combined organic phases were dried (Na₂SO₄), filtered, and concentrated in vacuo. Flash chromatography (SiO₂ , 3×15 cm, 4% MeOH—CHCl₃ eluent) afforded 6 (1.89 g, 6.21 mmol, 62%) as a white foam.

N-BOC-NMe-L--Cys(Me)-OH (7).

A solution of NMe-L-Cys-OH hydrochloride salt (5, 1.35 g, 10.0 mmol) in 100 mL of THF-H₂O (1:1) was sequentially treated with NaHCO₃ (1.68 g, 20.0 mmol) and Mel (0.65 mL, 10.5 mmol), and the reaction mixture was stirred at 25° C. for 3 h. The reaction mixture was brought to pH 8 by adding 1 N aqueous NaOH. Di-tert-butyl dicarbonate (2.62 g, 12.0 mmol) was added and the reaction mixture was stirred at 25° C. for 12 h maintaining a pH 8. The reaction mixture was poured onto 1 N aqueous HCl (150 mL) and extracted with CHCl₃ (3×100 mL). The combined organic phases were dried (Na_{2 SO}₄), filtered, and concentrated in vacuo. Flash chromatography (SiO₂, 3×15 cm, 2% MeOH—CHCl₃ eluent) afforded 7 (1.89 g, 7.63 mmol, 76%) as a colorless oil.

N-BOC-NMe-L-Cys(Me)-OMe (8).

Trimethylsilyl diazomethane (2.0 M hexane solution, 3.70 mL, 0.74 mmol) was added dropwise to a solution of 7 (1.86 g, 7.40 mmol) in 100 mL of benzene-MeOH (5:1) at 0° C. Following the addition, the reaction mixture was concentrated in vacuo. Flash chromatography (SiO₂, 3×15 cm, 20% EtOAc-hexane eluent) afforded 8 (1.77 g, 6.73 mmol, 91%) as a colorless oil.

NMe-L-Cys(Me)-OMe (9).

Compound 8 (1.32 g, 5.0 mmol) was treated with 5 mL of 3 M HCl-EtOAc and the mixture was stirred at 25° C. for 30 min before the volatiles were removed in vacuo. The residual HCl was removed by adding Et₂O (10 mL) to the hydrochloride salt followed by its removal in vacuo. The residue was dissolved in CHCl₃ (200 mL) and the organic layer was washed with saturated aqueous NaHCO₃ (100 mL) and saturated aqueous NaCl (100 mL). The organic phase was dried (Na₂SO₄), filtered, and concentrated in vacuo to give 9 (746 mg, 91%) as a colorless oil which was used directly in the next reaction without further purification.

(N-Cbz-D-Cys-OTce)₂ (10).

A solution of D-cystine (500 mg, 2.1 mmol) and NaOH (352 mg, 8.4 mmol) in 20 mL of THF-H₂O (1:1) was treated with CbzCl (0.63 mL, 4.4 mmol), and the reaction mixture was stirred at 25° C. for 1 h. The reaction mixture was diluted with water (50 mL) and washed with CHCl₃ (3×50 mL). The aqueous phase was acidified with 6 N aqueous HCl (50 mL) and extracted with CHCl₃ (3×50 mL). The combined organic phases were dried (Na₂SO₄), filtered, and concentrated in vacuo. The residue was dissolved in pyridine (20 mL), and HOBt (840 mg, 6.3 mmol) and trichloroethanol (0.69 mL, 5.3 mmol) were added. The mixture was cooled to −20° C., treated with DCC (1.29 g, 6.3 mmol), and the resulting mixture was stirred at −20° C. under Ar for 24 h. The white precipitate of DCU was removed by filtration, and the filtrate was concentrated in vacuo. The residue was diluted with EtOAc (100 mL), and the organic phase was washed with 1 N aqueous HCl (100 mL), saturated aqueous NaHCO₃ (100 mL), and saturated aqueous NaCl (50 mL). The organic phase was dried (Na₂SO₄), filtered, and concentrated in vacuo. Flash chromatography (SiO₂, 3×15 cm, 20% EtOAc-hexane eluent) afforded 10 (1.23 g, 1.6 mmol, 76%) as a colorless oil.

N-Cbz-D-Cys-OTce (11).

A solution of 10 (771 mg, 1.0 mmol) in 10 mL of THF was treated with Ph₃P (262 mg, 1.0 mmol), 2-mercaptoethanol (70 μL, 1.0 mmol), and water (180 μL, 10 mmol), and the reaction mixture was stirred at 50° C. for 5 h before being concentrated in vacuo. Flash chromatography (SiO₂, 3×18 cm, 20% EtOAc-hexane eluent) afforded 11 (764 mg, 1.98 mmol, 99%) as a colorless oil.

N-BOC-NMe-L-Cys(Acm)-NMe-L-Cys(Me)-OMe (12).

A solution of 6 (1.75 g, 5.74 mmol) in CH₂Cl₂ (57 mL) was treated sequentially with HOAt (781 mg, 5.74 mmol) and EDCl (1.10 g, 5.74 mmol), and the mixture was stirred at 0° C. for 15 min. A solution of 9 (935 mg, 5.74 mmol) was added and the reaction mixture was stirred for an additional 12 h. The reaction mixture was poured onto 1 N aqueous HCl (100 mL) and extracted with EtOAc (2×100 mL). The combined organic phases were washed with saturated aqueous NaHCO₃ (100 mL) and saturated aqueous NaCl (50 mL), dried (Na₂SO₄), filtered, and concentrated in vacuo. Flash chromatography (SiO₂, 3×15 cm, EtOAc eluent) afforded 12 (2.01 g, 4.46 mmol, 78%) as a white foam.

N-BOC-Gly-NMe-L-Cys(Acm)-NMe-L-Cys(Me)-OM (14).

A sample of 12 (2.01 g, 4.46 mmol) was treated with 4.5 mL of 3 M HCl-EtOAc and the mixture was stirred at 25° C. for 30 min before the volatiles were removed in vacuo. The residual HCl was removed by adding Et₂O (10 mL) to the hydrochloride salt 13 followed by its removal in vacuo. After repeating this procedure three times, 1.96 g of 13 (100%) was obtained and used directly in the following reaction without further purification.

A solution of N-BOC-Gly-OH (773 mg, 4.46 mmol) and hydrochloride salt 13 (1.96 g, 4.46 mmol) in CH₂Cl₂ (45 mL) was treated sequentially with HOAt (909 mg, 6.69 mmol), EDCl (1.26 g, 6.69 mmol), and NaHCO₃ (549 mg, 6.69 mmol), and the reaction mixture was stirred at 0° C. for 12 h. The reaction mixture was poured onto 1 N aqueous HCl (100 mL) and extracted with EtOAc (2×100 mL). The combined organic phase was washed with saturated aqueous NaHCO₃ (100 mL) and saturated aqueous NaCl (50 mL), dried (Na₂SO₄), filtered, and concentrated in vacuo. Flash chromatography (SiO₂, 5×14 cm, 20% acetone-EtOAc eluent) afforded 14 (1.54 g, 3.03 mmol, 68%) as a white foam.

N-BOC-Gly-NMe-L-Cys(Acm)-NMe-L-Cys(Me)-OH (15).

Lithium hydroxide monohydrate (92 mg, 2.31 mmol) was added to a solution of 14 (394 mg, 0.77 mmol) in 10 mL of THF-MeOH—H₂O (3:1:1) at 0° C. and the resulting reaction mixture was stirred at 25° C. for 1.5 h. The reaction mixture was poured onto 1 N aqueous HCl (100 mL) and extracted with CHCl₃ (3×50 mL). The combined organic phases were dried (Na₂SO₄), filtered, and concentrated in vacuo to give 15 (393 mg, 100%) as a white foam which was used without further purification.

N-Cbz-D-Cys[N-BOC-Gly-NMe-L-Cys(Acm)-NMe-L--Cys(Me)]-OTce (16).

A solution of 15 (393 mg, 0.77 mmol) in DMF (8 mL) was treated sequentially with HOAt (150 mg, 0.92 mmol) and EDCl (183 mg, 0.92 mnol), and the mixture was stirred at −20° C. for 15 min. A solution of 11 (300 mg, 0.77 mmol) was added and the reaction mixture was stirred for an additional 4 h. The reaction mixture was poured onto 1 N aqueous HCl (100 mL) and extracted with EtOAc (100 mL). The combined organic phase was washed with saturated aqueous NaHCO₃ (100 mL) and saturated aqueous NaCl (50 mL), dried (Na₂SO₄), filtered, and concentrated in vacuo. Flash chromatography (SiO₂, 3×15 cm, 33% EtOAc-hexane eluent) afforded 16 (551 mg, 0.64 mmol, 83%) as a white foam and epi-16 (28 mg, 0.032 mmol, 4%) as a white foam.

N-Cbz-D-Cys[N-Cbz-D-Cys(N-BOC-Gly-NMe-L-Cys(Acm)-NMe-L-Cys(Me))-Gly-NMe-L-Cys(Acm)-NMe-L--Cys(Me)]-OTce (19).

Compound 16 (432 mg, 0.5 mmol) was treated with 5.0 mL of 3 M HCl-EtOAc and the mixture was stirred at 25° C. for 30 min before the volatiles were removed in vacuo. The residual HCl was removed by adding Et₂O (10 mL) to the hydrochloride salt 17 followed by its removal in vacuo. After repeating this procedure three times, 429 mg of 17 (100%) was obtained and used directly in the following reaction without further purification.

A solution of 16 (432 mg, 0.5 mmol) in 90% aqueous AcOH (15 mL) was treated with Zn (1.62 g, 25 mmol) and the resulting suspension was stirred at 0° C. for 2 h. The zinc was removed by filtration and the filtrate was concentrated in vacuo. The residue was poured onto 1 N aqueous HCl (50 mL) and extracted with CHCl₃ (3×50 mL). The combined organic phase was dried (Na₂SO₄), filtered, and concentrated in vacuo to give 18 (430 mg, 100%) as a white foam which was employed directly in the next reaction without further purification. A solution of 17 (429 mg, 0.5 mmol) and 18 (430 mg, 0.5 mmol) in CH₂Cl₂ (5.0 mL) was treated sequentially with HOAt (98 mg, 0.6 mmol) and EDCl (119 mg, 0.6 mmol), and the reaction mixture was stirred at 0° C. for 6 h. The reaction mixture was poured onto 1 N aqueous HCl (50 mL) and extracted with EtOAc (2×50 mL). The combined organic phases were washed with saturated aqueous NaHCO₃ (50 mL) and saturated aqueous NaCl (30 mL), dried (Na₂SO₄), filtered, and concentrated in vacuo. Flash chromatography (SiO₂, 4×15 cm, 20% acetone-EtOAc eluent) afforded 19 (613 mg, 0.42 mmol, 83%) as a white foam.

N-Cbz-D-Cys[N-Cbz-D-Cys(N-BOC-Gly-NMe-L-Cys-NMe-L-Cys(Me)]-Gly-NM-L-Cys-NMe-L-Cys(Me)]-OH (21).

A solution of 19 (500 mg, 0.34 mmol) in 90% aqueous AcOH (15 mL) was treated with Zn (1.08 g, 17.0 mmol) and the resulting suspension was stirred at 0° C. for 2 h. The zinc was removed by filtration and the filtrate was concentrated in vacuo. The residue was poured onto 1 N aqueous HCl (100 mL) and extracted with CHCl₃ (3×50 mL). The combined organic phase was dried (Na₂SO₄), filtered, and concentrated in vacuo. The residue in CH₂Cl₂ (100 mL) was added dropwise to a solution of iodine (868 mg, 3.4 mmol) in 340 mL of CH₂Cl₂-MeOH (10:1) and the reaction mixture was stirred at 25° C. for 2 h. The reaction mixture was cooled in an ice bath and 5% aqueous Na₂S₂O₃ was added until the color of iodine disappeared. The mixture was washed with 1 N aqueous HCl (50 mL) and saturated aqueous NaCl (30 mL), dried (Na₂SO₄), filtered, and concentrated in vacuo. Flash chromatography (SiO₂, 3×16 cm, 10% MeOH—CHCl₃ eluent) afforded 21 (201 mg, 0.17 mmol, 49%, typically 49-53%) as a pale yellow foam.

[N-Cbz-D-Cys-Gly-NMe-L--Cys-NMe-L--Cys(Me)]₂ (Cysteine Thiol) Dilactone (23).

A sample of 21 (180 mg, 0.15 mmol) was treated with 1.5 mL of 3 M HCl-dioxane and the mixture was stirred at 25° C. for 30 min before the volatiles were removed in vacuo. The residual HCl was removed by adding Et₂O (5 mL) to the hydrochloride salt followed by its removal in vacuo. The residue in CH₂Cl₂ (150 mL) was treated sequentially with HOAt (122 mg, 0.75 mmol) and EDCl (149 mg, 0.75 mmol), and the reaction mixture was stirred at 0° C. for 6 h. The reaction mixture was poured onto 1 N aqueous HCl (50 mL) and extracted with EtOAc (2×50 mL). The combined organic phase was washed with saturated aqueous NaHCO₃ (50 mL) and saturated aqueous NaCl (30 mL), dried (Na₂SO₄), filtered, and concentrated in vacuo. Flash chromatography (SiO₂, 4×15 cm, 25% EtOAc-hexane eluent) afforded 23 (84 mg, 77 μmol, 52%, typically 52-61%) as a white solid.

Thi Coraline (1).

A sample of 23 (14.0 mg, 12.9 μmol) was treated with 2 mL of TFA-thioanisole (10:1) and the reaction mixture was stirred at 25° C. for 6 h before being concentrated in vacuo. The residue was treated with 3 M HCl-EtOAc and the volatiles were removed in vacuo to give the hydrochloride salt.

A solution of 25 (11.9 mg, 64.5 μmol) and DMAP (7.7 mg, 64.5 μmol) in CH₂Cl₂ (1 mL) was treated with EDCl (12.6 mg, 64.5 μmol) and the reaction mixture was stirred at 25° C. for 30 min. The hydrochloride salt 24 was added and the reaction mixture was stirred at 25° C. for 3 d. The reaction mixture was poured onto 1 N aqueous HCl (5 mL) and extracted with EtOAc (2×5 mL). The combined organic phases were washed with saturated aqueous NaCl (3 mL), dried (Na₂SO₄), filtered, and concentrated in vacuo. PTLC (SiO₂, CHCl₃:EtOAc:HOAc=10:20:0.3 eluent) afforded 1 (6.5 mg, 5.5 μmol, 43%) as a white solid which exhibited a ¹H NMR spectrum identical to the chart published for authentic 1 (Romeo, F., et al., J. Antibiot. 1997, 50, 734; Perez Baz, J., et al., J. Antibiot. 1997, 50, 738; Perez Baz, J., et al., PCT Int. Appl., WO952773, 1995; Chem. Abst. 1995, 124, 115561).

BE-22179 (2).

A sample of 1 (1.0 mg, 0.85 μmol) in 30% aqueous acetone (400 μL) was treated with NalO₄ (0.4 mg, 8.5 μmol) and the reaction mixture was stirred at 25° C. for 12 h before being quenched by adding aqueous Na₂S₂O₃. The mixture was concentrated in vacuo and the residue was extracted with EtOAc (2×2 mL). The combined organic phases were washed with saturated aqueous NaCl (3 mL), dried (Na₂SO₄), filtered, and concentrated in vacuo to give the crude sulfoxides. A solution of the crude sulfoxides in CH₂Cl₂ (400 μL) was warmed at reflux for 6 h and the volatiles were removed in vacuo. PTLC (SiO₂, CHCl₃:EtOAc:HOAc=10:20:0.3 eluent) afforded 2 (0.6 mg, 0.56 μmol, 66%) as a pale yellow solid which exhibited a ¹H NMR spectrum identical to the chart published for authentic 2 (Okada, H., et al., J. Antibiot 1994, 47, 129).

[N-(2-Quinoline Carboxyl)-D-Cys-Gly-NMe-L--Cys-NMe-L--Cys(Me)]₂ (Cysteine Thiol) Dilactone (26).

In the manner described for 1, the reaction of 23 (5.0 mg, 4.6 μmol) with quinoline-2-carboxylic acid (4.0 mg, 23.0 μmol), EDCl (4.5 mg, 23.0 μmol), and DMAP (2.8 mg, 23.0 μmol) in CH₂Cl₂ (300 μL) and purification by PTLC (SiO₂, CHCl₃:EtOAc:HOAc=10:20:0.3 eluent) afforded 26 (2.8 mg, 2.4 μmol, 52%) as a white foam.

[N-(2-Quinoxaline carboxyl)-D-Cys-Gly-NMe-L--Cys-NMe-L--CYS(Me)]₂ (Cysteine Thiol) Dilactone (27).

In the manner described for 1, the reaction of 23 (5.0 mg, 4.6 μmol) with quinoxaline-2-carboxylic acid (4.0 mg, 23.0 μmol), EDCl (4.5 mg, 23.0 μmol), and DMAP (2.8 mg, 23.0 μmol) in CH₂Cl₂ (300 mL) and purification by PTLC (SiO₂, CHCl₃:EtOAc:HOAc=10:20:0.3 eluent) afforded 27 (2.0 mg, 2.2 μmol, 47%) as a white foam.

[N-(3-Hydroxy-6-methoxy-2-quinoline carboxyl)-D-Cys-Gly-NMe-L--CyS-NMe-L--Cys(Me)]₂ (Cysteine Thiol) Dilactone (28).

In the similar manner described for 1, the reaction of 23 (5.0 mg, 4.6 μmol) with 3-hydroxy-6-methoxy-quinoline-2-carboxylic acid (Isolation: Konishi, M., et al., J. Antibiot. 1981, 34, 148. Structure and stereochemistry: Arnold, E., et al., J. Am. Chem. Soc. 1981, 103, 1243; Total synthesis (luzopeptins A-C): Boger, D. L., et al., J. Am. Chem. Soc: 1999, 121, 1098; Boger, D. L., et al., J. Am. Chem. Soc. 1999, 121, 11375; Luzopeptin E2: Ciufolini, M. A., et al., J. Heterocyclic Chem. 1999, 36, 1409; Ciufolini, M. A., et al., Angew. Chem., Int. Ed. 2000, 39, 2493; Boger, D. L., et al., J. Org. Chem. 1995, 60, 7369) (4.0 mg, 23.0, μmol), EDCl (4.5 mg, 23.0 μmol), and DMAP (2.8 mg, 23.0 mmol) in CH₂Cl₂ (300 μL) and purification by PTLC (SiO₂, CHCl₃:EtOAc:HOAc=10:20:0.3 eluent) afforded 28 (2.5 mg, 2.4 μmol, 51%) as a white foam.

### DETAILED DESCRIPTION OF FIGURES

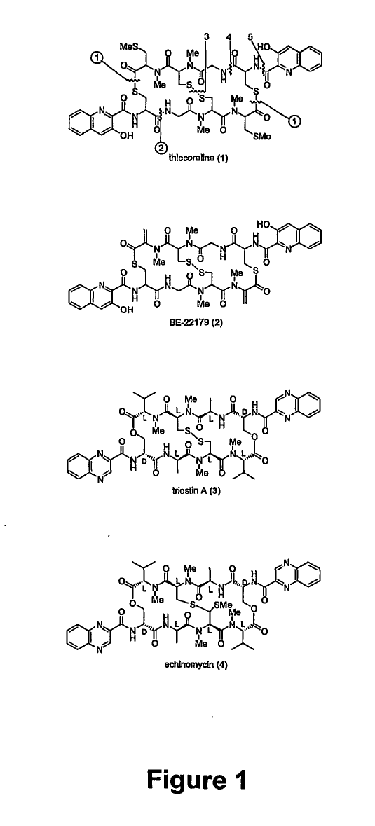

FIG. 1 shows the structures of thiocoraline (1), BE-22179 (2), triostin A (3) and echinomycin (4). Thiocoraline is a potent antitumor antibiotic isolated from Micromonospora sp. L-13-ACM2-092. It constitutes the newest member of the two-fold symmetric bicyclic octadepsipeptides which include the antitumor antibiotics BE-22179 (2), triostin A (3), and echinomycin (4), which bind to DNA with bisintercalation.

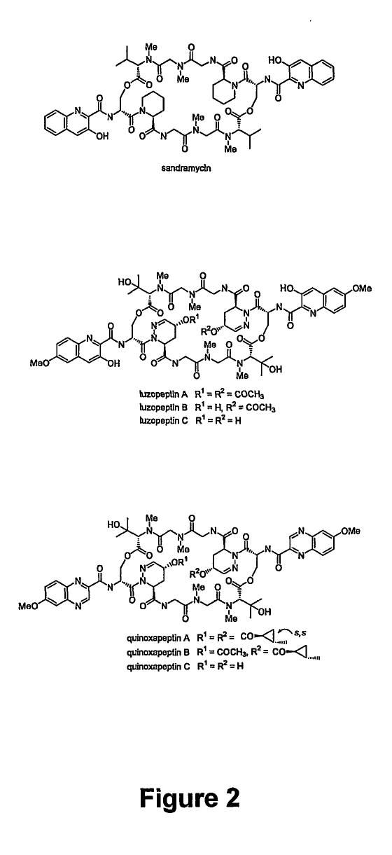

FIG. 2 shows the structures of members of the larger cyclic decadepsipeptides including sandramycin, the luzopeptins, and the quinoxapeptins. Triostin A and echinomycin possess a D-stereochemistry at the a-position of the amide linkage to the quinoxaline chromophore (D-Ser) and L-stereochemistry at the remaining stereogenic centers. The analogous centers of sandramycin and the quinoxapeptins like the luzopeptins, also incorporate D-Ser.

FIG. 3 is a scheme showing a convergent assemblage of key tetradepsipeptide 16 from tripeptide 15 and N-Cbz-D-Cys-OTce (11) along with the preparation of the three suitably functionalized Cys residues found in 1. Sequential S- and N-protection of N-Me-Cys-OH (5) with an acetamidomethyl (Acm) group (1.5 equiv of N-hydroxymethylacetamide, H₂SO₄) and BOC group (BOC₂O, 62%) gave 6, the precursor to the bridging disulfide Cys residue. Selective S-methylation of N-Me-Cys-OH (5), Mel, NaHCO₃) followed by BOC protection (BOC₂O, NaOH, 73%) provided 7. Esterification of 7 (TMSCHN₂, 89%) followed by BOC deprotection of 8 (3 M HCl-EtOAc, 91%) provided 9, the precursor to the second functionalized L-Cys residue. Compound 11, constituting the chromophore bearing D-Cys residue, was prepared by the reduction of its disulfide precursor 10 (Ph₃P, 2-mercaptoethanol, 99%) which in turn was obtained by stepwise Cbz (CbzCl, NaHCO₃) and Tce (trichloroethanol, DCC, (DCC=dicyclohexylcarbodiimide; EDCl=1-(3-dimethylaminopropyl)-3-ethylcarbodiimide hydrochloride; HOBt=1-hydroxy-benzotriazole; HOAt=1-hydroxy-7-azabenzotriazole) HOBt, 76%) protection of D-cystine. The esterification reaction with trichloroethanol proved sensitive to racemization and when conducted in the absence of HOBt (33% de vs 100% de) or in the presence of DMAP (33% de) led to extensive racemization. Coupling of 6 with 9 (EDCl, HOAt, 78%) provided 12 and slightly lower conversions was obtained with HOBt vs HOAt. BOC deprotection of 12 (3 M HCl-EtOAc, 100%), coupling with N-BOC-Gly-OH (EDCl, HOAt, 68%) and methyl ester hydrolysis of 14 (LiOH, 100%) provided 15. The key thiol esterification reaction linking the D-cysteine derivative 11 and the tripeptide 15 was accomplished under near racemization free conditions with use of EDCl-HOAt (83%) in the absence of added base to afford the depsipeptide 16 (de 95:5).

FIG. 4 is a scheme for the synthesis of 2, 26, 27 and 28. The starting amine 17 and the free acid 18 were mixed in the absence of added base (EDCl, HOAt, CH₂Cl₂, 83%) to obtain 19 (FIG. 4). Cyclization of 19 to provide the 26-membered cyclic octadepsi-peptide 23 with ring closure conducted at the single secondary amide site was accomplished by sequential Tce ester deprotection (Zn, 90% aq. AcOH), disulfide bond formation (12, CH₂Cl₂-MeOH, 25° C., 0.001 M, 53% for 2 steps), and BOC deprotection (3 M HCl-dioxane) followed by treatment with EDCl-HOAt (0.001 M CH₂Cl₂, −20° C., 6 h, 61% for 2 steps). Reversing the N-BOC deprotection and disulfide bond formation steps in this 4-step sequence resulted in lower conversions (13% overall for 4 steps). To date, all attempts to effect ring closure followed by disulfide bond formation have not been successful. Even though the 26-membered ring macrocyclization reaction unconstrained by the disulfide bond proceeds exceptionally well (>50%), the subsequent disulfide bond formation (I₂, CH₂Cl₂-MeOH, 25° C.) within the confines of the 26-membered ring failed to occur. Thus, the order of steps enlisted for formation of 23 was not to improve macrocyclization via the constrained disulfide, but rather to permit disulfide bond formation. While it is possible this may be due to constraints within the macrocycle destabilizing the disulfide, the lack of similar observations with 3 and 4 suggest the origin of the difficulties may lie with competitive intramolecular cleavage of the adjacent thiol ester by the liberated bridging thiol within the 26-membered macrocycle.

FIG. 5 is a scheme showing the successful synthesis of 32. Tetradepsipeptide 30 and octadepsipeptide 31 were prepared by the procedures described for the synthesis of 16 and 19. Cyclization of 31 to provide the bridged 26-membered cyclic octadepsipeptide 32 was accomplished by sequential Tce ester deprotection (Zn, 90% aq. AcOH), BOC deprotection (3 M HCl-dioxane), and disulfide bond formation (I₂, CH₂Cl₂-MeOH, 25° C., 0.001 M) followed by treatment with EDCl-HOAt (0.001 M CH₂Cl₂, −20° C., 6 h, 16/o for 4 steps).

However, exposure of 32 to Et₂NH or piperidine led to decomposition of the macrocycle rather than clean FMOC deprotection. Alternative treatment of 32 with other amines including dicyclohexylamine, Et₃N, or DMAP also failed to provide the cyclic amine 24 which were attributed to the sensitivity of the thiol ester to nucleophiles, the competitive b-elimination induced by the deprotonation of the a-position of the Cys residues, and a potential intramolecular S—N acyl transfer to the liberated amine with cleavage of the thiol ester. However, efforts to trap the liberated amine in situ to obtain 1 directly (25, EDCl, DMAP) or a protected derivative of 24 (BOC₂₀ or CbzCl, Et₃N) were also unsuccessful.

FIG. 6 shows an approach in which the pendant chromophore was introduced at the initial stages of the synthesis. Thus, the coupling reaction of 15 and 34 (EDCl, HOAt, 86%) gave tetradepsipeptide 35 which possesses the substituted quinoline chromophore.

FIG. 7 shows two plots of fluorescence vs. the DNA to drug ratio and the resulting Scatchard plot for each. Scatchard analysis (Scatchard, G. Ann. N.Y. Acad. Sci. 1949, 51, 660) of the titration results was conducted using the equation r_{b}/c=Kn−Kr_{b}, where r_{b} is the number of molecules bound per DNA nucleotide phosphate, c is the free drug concentration, K is the apparent binding constant, and n is the number of the agent binding sites per nucleotide phosphate. A plot of r_{b}/c versus r_{b} gives the association constant (slope) and the apparent binding site size (x-intercept) for the agents. (a) Fluorescence quenching of thiocoraline (excitation at 380 nm and emission at 510 nm in Tris-HCl (pH 7.4) and 75 mM NaCl buffer solution) with increasing CT-DNA concentration. (b) Scatchard plot of fluorescence quenching of part a. (c) Fluorescence quenching of BE-22179 (excitation at 380 nm and emission at 510 nm in Tris-HCl (pH 7.4) and 75 mM NaCl buffer solution) with increasing CT-DNA concentration. (d) Scatchard plot of fluorescence quenching of part c.

FIG. 8 is a table of comparative DNA binding properties. ^{a}Calf thymus DNA, KB=apparent binding constant determined by fluorescence quenching. The value in paren-theses is the agent/base pair ratio at saturated high-affinity binding and may be considered a measure of the selectivity of binding. ^{b}Agent/base pair ratio required to unwind negatively supercoiled FX174 DNA (form I to form II gel mobility, 0.9% agarose gel). ^{c}Agent/base pair ratio required to induce complete rewinding or positive super-coiling of FX174 DNA (form II to form I gel mobility, 0.9% agarose gel). ^{d}Binding constant established by footprinting at a 5′-CCGC site (FIG. 9).

FIG. 9 is an electrophoresis gel of DNase footprinting of echinomycin bound to w794 DNA. Lane 13, G, C and A Sanger sequencing reactions; lane 4, native DNA; lane 5, control DNA without treatment of DNase I; lanes 6-14; 0, 10, 20, 40, 60, 80, 100, 120, and 140 mM echinomycin with DNase I treatment (1 min).

FIG. 10 is an electrophoresis gel of DNase footprinting of thiocoraline bound to w794 DNA. Lane 1, native DNA; lane 2, control DNA without treatment of DNase I; lanes 3-6, G, C, A, and T Sanger sequencing reactions; lanes 7-26; 0, 10, 25, 50, 75, 100, 150, 200, 250, 300, 350, 400, 450, 500, 600, 700, 800, 900, 1000, and 0 mM thiocoraline with DNase I treatment (1 min).

FIG. 11 shows a series of three electrophoresis agarose gels in which thiocoraline, echinomycin, BE-22179, and 27 are tested for their ability to uncoil DNA. (A) Lane 1, untreated supercoiled FX174 DNA, 95% form 1 and 5% form II; lanes 2-8, thiocoraline-treated FX174 DNA; lanes 9-14, echinomycin-treated FX174 DNA. The [agent]-to-[base pair] ratios were 0.022 (lanes 2 and 9), 0.033 (lanes 3 and 10), 0.044 (lanes 4 and 11), 0.066 (lanes 5 and 12), 0.11 (lanes 6 and 13), 0.22 (lanes 7 and 14), and 0.44 (lane 8). (B) Lane 1, untreated supercoiled FX174 DNA, lanes 2-12, BE-22179-treated FX174 DNA. The [agent]-to-[base pair] ratios were 0.022, (lane 2), 0.033 (lane 3), 0.044 (lane 4), 0.066 (lane 5), 0.11 (lane 6), 0.22 (lane 7), and 0.33 (lane 8), 0.44 (lane 9), 0.66 (lane 10), 1.1 (lane 11), and 2.2 (lane 12). (C) Lane 1, untreated supercoiled FX174 DNA, 95% form 1 and 5% form II; lanes 2-8, thiocoraline analogue (27)-treated FX174 DNA. The [agent]-to-[base pair] ratios were 0.022 (lane 2), 0.033 (lane 3), 0.044 (lane 4), 0.066 (lane 5), 0.11 (lane 6), 0.22 (lane 7), and 0.44 (lane 8).

FIG. 12 is a table showing that thiocoraline binds to DNA with high affinity, but with little or no selectivity. The binding of 1 was examined with a set of four duplex deoxyoligonucleotides, 5′-GCXXGC-3′ where XX=TA, AT, GC, CG, incorporating the high affinity intercalation sites of the related bisintercalatiors echinomycin (5′-PuCGPy), sandramycin (5′-CATG), and the luzopeptins (5′-CATG). A small preference was observed for GC-rich binding with 5′-GCGCGC and 5′-GCCGGC exhibiting the tightest binding, but the differences are small ranging from 3-7×10⁶ M⁻¹ for the four deoxyoligonucleotides. Thus, consistent with the results of footprinting and other related studies herein, the binding of 1 with the deoxyoligonucleotides exhibited little selectivity.

FIG. 13 summarizes the biological properties of echinomycin, thiocoraline, and BE-22179 along with those of precursor 23 and their analogues. Thiocoraline and BE-22179 exhibit exceptionally potent cytotoxic activity in the L1210 assays (IC₅₀=200 and 400 pM, respectively) being slightly less potent than echinomycin. Compounds 23 and 32 lacking both chromophores and containing the Cbz and FMOC protecting groups were inactive and >10⁵ times less potent than thiocoraline. Analogue 28, which bears the same chromophore as the luzopeptins, also exhibited potent activity while 26, lacking the quinoline C3 phenol, and 27, bearing the quinoxaline chromophore of echinomycin and triostin A, exhibited less potent cytotoxic activity. In addition, thiocoraline, like echinomycin, was found to be only a weak inhibitor of HIV-1 reverse transcriptase.

What is claimed is:

1. A compound represented by the following structure: 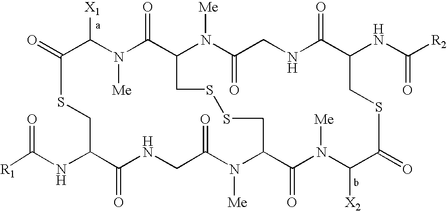
wherein:
X₁ and X₂ are selected from the group consisting of ═CH₂ and —CH₂SMe; and
R₁ and —R₂ are selected from the group consisting of hydrogen, Cbz, FMOC, and radicals represented by the following structure: 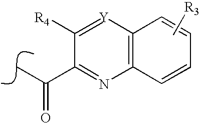
wherein;
Y is selected from the group consisting of C and N;
R₃ is either absent or —O(C1-C6 alkyl); and
R₄ is selected the group consisting of hydrogen and hydroxyl; with the following provisos:
if X₁ is ═CH₂, then “a” represents a double bond and neither R₁ nor R₂ is hydrogen;
if X₁ is —CH₂SMe, then “a” represents a single bond;
if X₂ is ═CH₂, then “b” represents a double bond and neither R₁ nor R₂ is hydrogen;
if X₁ is —CH₂SMe, then “b” represents a single bond; and
if R₃ is absent, then Y is N or R₄ is hydrogen.

2. A compound according to claim 1 represented by the following diastereomeric structure:

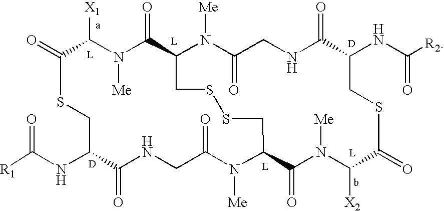

3. A compound according to claim 2 represented by the following diastereomeric structure:

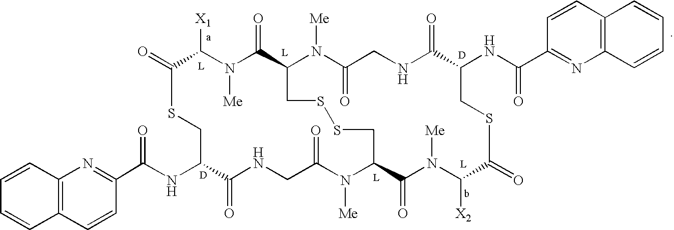

4. A compound according to claim 3 represented by the following diastereomeric structure:

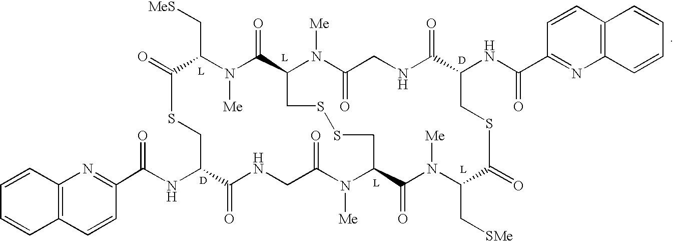

5. A compound according to claim 3 represented by the following diastereomeric structure:

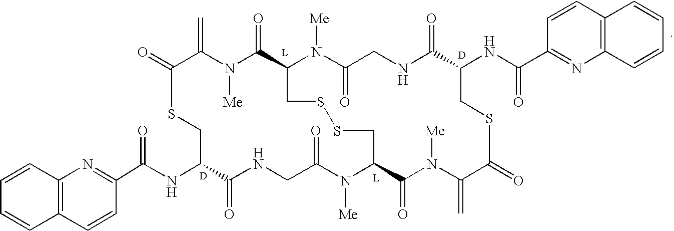

6. A compound according to claim 2 represented by the following diastereomeric structure:

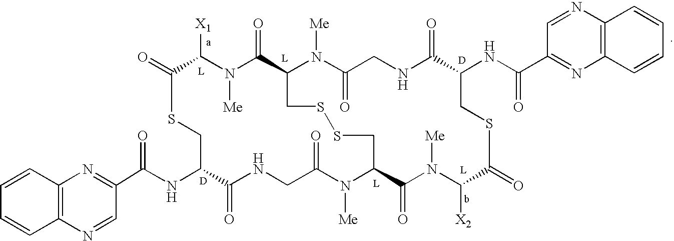

7. A compound according to claim 6 represented by the following diastereomeric structure:

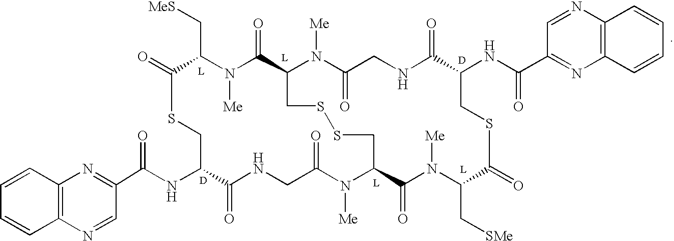

8. A compound according to claim 6 represented by the following diastereomeric structure:

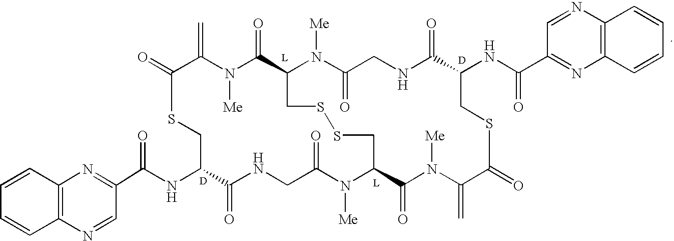

9. A compound according to claim 2 represented by the following diastereomeric structure:

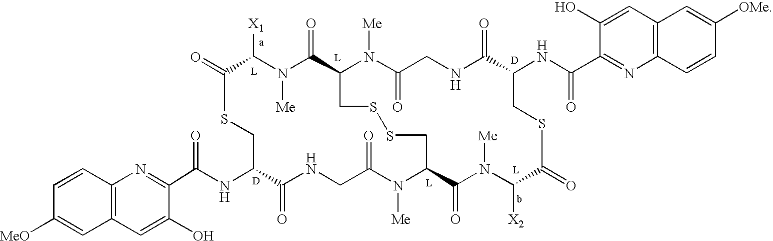

10. A compound according to claim 9 represented by the following diastereomeric structure:

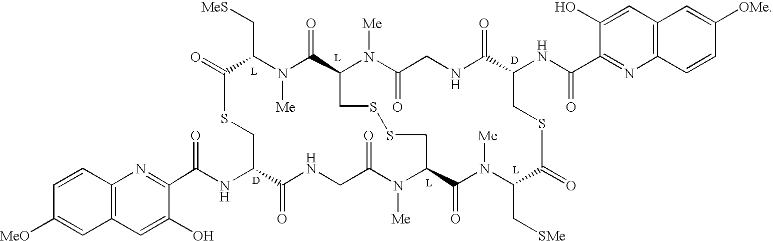

11. A compound according to claim 9 represented by the following diastereomeric structure:

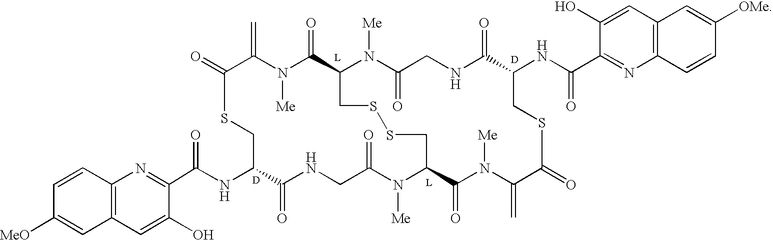

12. A compound according to claim 2 represented by the following diastereomeric structure:

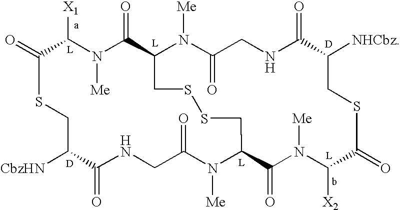

13. A compound according to claim 12 represented by the following diastereomeric structure:

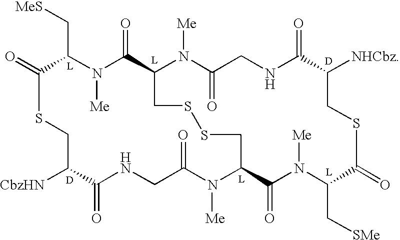

14. A compound according to claim 12 represented by the following diastereomeric structure:

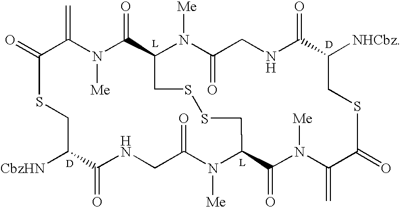

15. A compound according to claim 2 represented by the following diastereomeric structure:

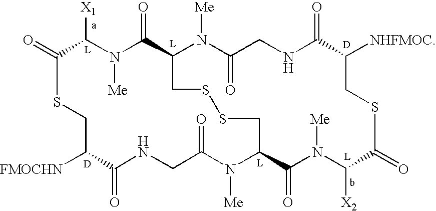

16. A compound according to claim 15 represented by the following diastereomeric structure:

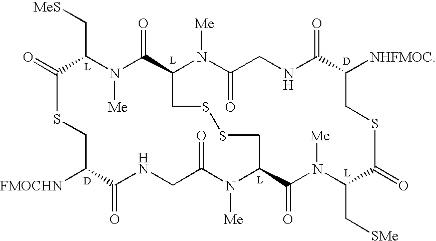

17. A compound according to claim 15 represented by the following diastereomeric structure:

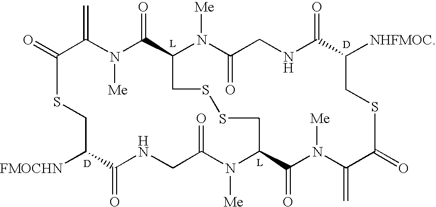

18. A compound according to claim 2 represented by the following diastereomeric structure:

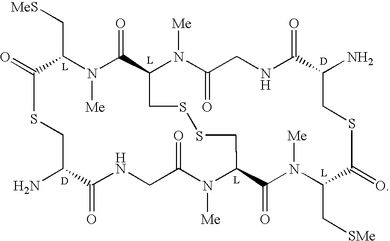

19. A process for killing a cancer cell comprising the step of contacting said cancer cell with a composition containing a concentration of thiocoraline, BE-22179, or a compound described in any of claims 1-18, said concentration being sufficient to be cytotoxic with respect to said cancer cell.

20. A process for binding thiocoraline, BE-22179, or a compound described in any of claims 1-18 to a deoxyoligonucleotide or a deoxypolynucleotide, said process comprising the step of binding said thiocoraline, BE-22179, or compound described in any of claims 1-18 to said deoxyoligonucleotide or said deoxypolynucleotide by bisintercalation.

21. A process for synthesizing an advanced intermediate comprising the following step:
Cyclizing a first intermediate represented by the following structure: 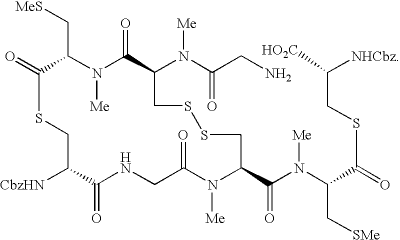
for producing the advanced intermediate represented by the following structure: [chemical structure]
